(19) United States
(12) Patent Application Publication
(10) Pub. No.: US 2011/0089118 A1
(43) Pub. Date: Apr. 21, 2011

(54) SURFACE-ROUGHENED HIGH-DENSITY FUNCTIONAL PARTICLE, METHOD FOR PRODUCING THE SAME AND METHOD FOR TREATING TARGET SUBSTANCE WITH THE SAME

(76) Inventors: Naoki Usuki, Osaka (JP); Masakazu Mitsunaga, Osaka (JP); Kenji Kohno, Osaka (JP); Hisao Kanzaki, Osaka (JP)

(21) Appl. No.: 12/999,930

(22) PCT Filed: Apr. 24, 2009
(86) PCT No.: PCT/JP2009/058603

§ 371 Date: Dec. 17, 2010

(30) Foreign Application Priority Data
Jun. 18, 2008 (JP) 2008-158949

(51) Int. Cl. B01D 21/01; B32B 3/00

(52) U.S. Cl. 210/714; 428/402; 216/83

(57) ABSTRACT

The particle of the present invention is a high-density particle to which a target substance can be bound, wherein the surface of the particle body is a roughened surface. The particle is characterized in that a substance or functional group to which a target substance can bind is immobilized on the roughened surface of the particle body, and the specific surface area of the particle is 1.4 to 100 times the specific surface area of a true spherical particle having the same particle size and the same density as those of the particle of the invention. In the particle of the invention, the accumulated micropore volume [cm³] of micropores having radius of not less than 20 nm per unit surface area [cm²] is not less than 1×10⁻⁶ [cm³/cm²].

### TECHNICAL FIELD

The present invention relates to a functional particle having a roughened surface with a specific surface area suited for a separation, immobilization, analysis, extraction, purification, reaction or the like of a target substance. The present invention also relates to a method for producing the above particle, and further relates to a method for treating a target substance by using of the above particle.

### BACKGROUND OF THE INVENTION

Composite particles capable of specifically binding to or reacting with particular kinds of target substances have conventionally been well known as functional materials for use in biochemical applications. Examples of such applications using the particles include a quantitative determination, a separation, a purification and an analysis of the target substances (e.g. cells, proteins, nucleic acids and chemical substances). See Patent Document 1: Japanese Patent Kokai Publication No. 4-501956. The above composite particles are magnetized particles which are for example produced by incorporating a magnetic material into nonmagnetic beads. When the composite particles are used for the purpose of separating target substances from a sample, the composite particles are added to the sample containing the target substances in order to allow the target substances to bind to the surfaces of composite particles. Subsequently, a magnetic field is applied in order to allow the composite particles to assemble and aggregate in the sample. By collecting and recovering the assembled and aggregated composite particles, the target substance together with the composite particles can be separated. This method makes use of the magnetic field or magnetism (the method using the magnetic field or magnetism hereinafter can be also referred to as “magnetic separation method” or simply referred to as “magnetic separation”). Therefore, this method has such a feature that it can be carried out even if the amount of the sample is smaller than the amount intended for use in a centrifugal separation method, a column separation method, an electrophoresis method or the like, and also it can be carried out in a short time without causing a denaturation of the target substances. However, the above composite particles have a small density of 1.0 g/cm³ to 3.4 g/cm³, and thus such composite particles is not suited for achieving an efficient aggregation of the particles. The reason for the comparatively small density of the composite particles is that they are prepared from a low-density resin or silica serving as base material and a magnetic powder material dispersed therein. In other words, considering that the density of the composite particles depends on the amount of the magnetic powder material, the content of such magnetic powder material is only about 20% by weight at most when calculated from the magnetization amount, and therefore the density of the composite particles is more or less close to the low density of the base material, i.e. the low-density resin or silica.

Considering that the target substance can bind to the surfaces of the particles, the binding amount of the target substance with respect to a single particle depends on the specific surface area of the particle. That is, if the particle has a small specific surface area, the binding amount of the target substance to the single particle will decrease. Such decrease in the binding amount of the target substance can cause a reduction of the detected amount of the target substances as a whole upon detection thereof, which will lead to a decreased sensitivity for detection of the target substances. For this reason, it is preferred that the specific surface area of the particle is large to some extent. However, the larger specific surface area of the particle is not necessarily better. In this regard, when the particle has a three-dimensional interpenetrating network structure (i.e. through-pore) or has deep holes, the target substance fails to enter the pore, or a desirable reaction fails to proceed even if the target substance can enter the pore. In addition, there is possibility to increase an apparent “nonspecific binding” in which a substance other than the target substance is hard to escape from the pores after entered. That is, in the case where the specific surface area of the particle is too large beyond necessity, the target substance cannot enter the pores, and thus the large specific surface area is not effectively available. Moreover, the too large specific surface area is not preferable since the effect of “nonspecific binding” in which a substance other than the target substance binds to the particle becomes great. For example, Patent Document 2 (Japanese Patent Kokai Publication No. 9-503989) discloses an example using a high-density zirconia particle. However, the zirconia particle disclosed in Patent Document 2 is a porous particle having a three-dimensional interpenetrating network (namely, through-pore), and thus a nonspecific binding phenomenon is likely to occur beyond necessity upon the separation of the target substance, due to an extremely large specific surface area of the particle. In other words, in the zirconia particle with the through-pores therein as disclosed in Patent Document 2, the substances other than the target substances tend to easily bind to the zirconia particles, and thus the target substances are hard to preferentially bind to the particles, which will prevent an achievement of the separation of the target substances. Furthermore, the zirconia particle as disclosed in Patent Document 2 will lead to an increase in the apparent nonspecific binding wherein the target substance is trapped in the through-pores or deep pores so that it is hard to escape therefrom.

Incidentally, Patent Document 2 describes about “pore size”. Patent Document 2 also describes that “the pore size of the particle is sufficient to accommodate proteins serving as the ligand or the adsorbed agent”. However, it is silent on the matter as to which poresize (i.e. which radius of the micropore) and how much volume (i.e. how much accumulated volume of micropore) the particle should have.

### DISCLOSURE OF THE INVENTION

Under these circumstances, the present invention has been created. In other words, an object of the present invention is to provide a particle which is suited for the separation of a target substance in terms of not only a movement and aggregation of the particles but also a nonspecific binding. Another object of the present invention is to provide a method for producing such particle and also provide a method for performing an analysis, extraction, purification or reaction of a target substance by the use of the particles of the present invention.

In order to achieve the above objects, the present invention provides “particle to which a target substance can bind”, characterized in that:

“substance or functional group capable of binding to the target substance” is immobilized on a surface of a particle body thereof; and

the surface of the particle body is a roughened surface and a specific surface area of the particle is 1.4 to 100 times a specific surface area of a true spherical particle having the same particle size and the same density as those of the particle of the invention.

As used in this description and claims, the term “roughened” substantially means that the particle has been subjected to a treatment for increasing the surface area of the particle (more specifically, “surface area of the particle body”).

Preferably, due to “roughened”, the particles have a ratio of an accumulated micropore volume [cm³] of micropores having radius of not less than 20 nm per unit surface area [cm²] is not less than 1×10⁻⁶ [cm³/cm²]. As used in this description and claims, the term “micropore” substantially means a void of the particle, more preferably the void existing in the vicinity of the surface of the particle, which includes a macropore having a diameter (size) ranging from 100 nm to 10 μm and a mesopore having a diameter (size) ranging from 1 nm to 100 nm. In particular, the term “micropore” corresponds to the mesopore with a diameter of 1 nm to 100 nm. Such macropore and mesopore can be simultaneously determined by a mercury intrusion technique.

The particle of the present invention has “substance or functional group capable of binding to a target substance” immobilized thereon. In other words, “substance or functional group to which a target substance can bind” is immobilized on the particle. Therefore, when the target substance and particle coexist with each other, the target substance can bind to the particle. Therefore, the particles of the present invention can be used for not only various applications such as separation, purification and extraction of the target substance, but also applications of tailor-made medical technologies. As used in this description and claims, the term “target substance” substantially means an object substance in various applications such as separation, extraction, quantitative determination, purification and analysis. “Target substance” may be any suitable substances as long as it can bind to the particle directly or indirectly. Examples of the target substance include nucleic acids, proteins (e.g. avidin, biotinylated HRP and the like), sugars, lipids, peptides, cells, eumycetes(fungus), bacteria, yeasts, viruses, glycolipids, glycoproteins, complexes, inorganic substances, vectors, low molecular compounds, high molecular compounds, antibodies and antigens. The particles of the present invention have various functions, considering that they can be used for separation, purification, extraction and analysis of various target substances. It should be therefore noted that the particles of the present invention can be called “functional particles”.

The particle of the present invention is characterized in that it has been subjected to a surface-roughening treatment. Specifically, the body of the particle has a roughened surface, and thus the particle has a specific surface area which is 1.4 to 100 times the specific surface area of the true spherical particle (i.e. a spherical particle with its smoothed surface) having the same particle size and the same density as those of the particle of the invention. With respect to the particle of the invention, a ratio of the accumulated volume [cm³] of micropores having radius of not less than 20 nm per unit surface area [cm²] is not less than 1×10⁻⁶ [cm³/cm²]. As used in this description and claims, the term “true spherical particle” substantially means a particle whose shape is a true sphere in terms of geometry. The term “true sphere” means a sphere wherein all the diameters passing through the center of the sphere have substantially the same length. In particular, the phrase “true spherical particle having the same particle size” means a true spherical particle which has a smoothed surface or even surface as a whole, and has the same particle size as that of the present particle. In this context, the surface of the particle of the present invention is in a roughened form due to the roughening treatment. Thus, as the particle size of the present invention, a diameter of a true circle is substantially available, which circle has the same area as a particle area that is obtained from the number of the pixels in a particle image of an electron micrograph or an optical micrograph (the particle area may include the thickness of a polymer coating that covers the particle body, if any). In this regard, the value of the specific surface area is usually obtained as a mean value of a plurality of particles. Thus, for example, the particle size can be used as an average particle size which is obtained by measuring each particle size of for example 300 particles based on the image and then calculating the number average thereof. In the measurement of the (average) particle size from the image, an image processing software (e.g. “Image-Pro Plus” manufactured by Media Cybernetics, Inc.) can be used. Summarizing the above matters, the phrase “specific surface area of a true spherical particle having the same particle size and the same density as those of the particle of the present invention” substantially means a specific surface area of the true spherical particle which has a diameter D corresponding to an average diameter L of the true circle having the same area as the area of the image on the particles of the present invention wherein the density of the true spherical particle is the same as the density of the particle of the present invention.

The particle of the present invention is characterized by not only an increased specific area resulted from the surface-roughening treatment, but also a predetermined amount or more of the desired-sized micropores existing on the surface thereof. Specifically, the particle of the present invention is characterized in that it has a predetermined amount or more of micropores which are larger in size than that of “substance or functional group capable of binding to a target substance” as well as “target substance”. Accordingly, as for the particle of the present invention, more number of “substance or functional group capable of binding to a target substance” can be immobilized thereon, and more number of target substances can bind to the “substance or functional group capable of binding to a target substance” when the particle of the present invention is in use.

The latter case will be explained in detail. When the particle is in use, the target substance can bind to the “substance or functional group capable of binding to a target substance” immobilized on the particle body. As for the “substance or functional group capable of binding to a target substance” immobilized within the micropores of the particle, the target substance enters the micropores and then binds to the “substance or functional group capable of binding to a target substance”. That is, it is contemplated that the larger-sized target substance cannot enter the smaller-sized micropores, so that the larger-sized target substance cannot bind to the “substance or functional group capable of binding to a target substance” existing inside the micropores. In this regard, however, the particle of the present invention has an effect in that an increased binding amount of the target substance is expected when the particle is in use, since the particle of the present invention has a predetermined amount or more of larger-sized micropores compared to the size of “substance or functional group capable of binding to a target substance” as well as the size of “target substance”.

In another aspect, the particle of the present invention is characterized in that no compound derived from an acidic substance that was used in the surface-roughening treatment (specifically, “compounds containing the metal element and the acidic substance”) substantially adheres to or remains on the surface of the particle body. That is, none of hydrochloric acid compound, sulfuric acid compound and nitric acid compound, which are derived from at least one acidic substance selected from the group consisting of hydrochloric acid, sulfuric acid and nitric acid, substantially adheres to or remains on the surface of the particle body.

The particle of the present invention preferably has a density of 3.5 g/cm³ to 9.0 g/cm³. This means that the particle of the present invention has the density (or specific gravity) higher than that of a general particle used commonly for separation of the target substance.

In the particle of the present invention, the particle body may have through-pores or no through-pore. In this regard, the expression “particle body has no through-pore” means that the body of the particle is substantially solid and thus the particle has no “interpenetrating network structure”. That is to say, the phrase “particle body has no through-pore” has the same meaning as “particle body or core portion thereof is solid”, “even if the particle has a rough surface, there is no recess existing in the interior of the particle” and “the bulk density of the particle is higher as compared with that of a conventional porous particle”. In the case where the particle body has no through-pore, the “effect of decreasing the nonspecific adsorptivity”, which will be described later, is remarkably expected.

The present invention also provides a method of producing the above particle. Such method is a method for producing a particle to which a target substance can bind comprising:

(I) contacting the precursor particle with at least one kind of acidic substance selected from the group consisting of hydrochloric acid, sulfuric acid and nitric acid (excluding phosphoric acid); and

(II) immobilizing the “substance or functional group capable of binding to a target substance” to the precursor particle.

The producing method of the present invention is characterized in that, during the step (I), the surface of the precursor particle is roughened so that a specific surface area of the resulting roughened particle is 1.4 to 100 times the specific surface area of a true spherical particle which has the same particle size and the same density as those of the present particle. In a more preferred embodiment, the surface of the precursor particle is roughened so that the ratio of the accumulated volume [cm³] of micropores having radius of not less than 20 nm per unit surface area [cm²] of the particle or particle body is not less than 1×10⁻⁶ [cm³/cm²]. In a further preferred embodiment, the surface of the precursor particle is roughened so that the resulting particle or particle body has no through-pore. In this case, a particle with no through-pore inside thereof can be finally obtained. In the method of the present invention, it is preferable to use a precursor particle with its density ranging from 3.5 g/cm³ to 9.0 g/cm³ for the roughening treatment in order that the obtained particle has a density (or a specific gravity) higher than that of a general particle commonly used for separation of the target substance. Furthermore, the production method of the present invention is characterized in that, after the step (I) (and also after washing treatment of the resulting particle if necessary), none of compounds which is derived from the acidic substance used in the surface-roughening treatment (particularly “compounds containing the metal element and the acidic compound”) substantially adheres to or remains on the surface of the particle body. In other word, the surface of the particle body substantially has no hydrochloric acid compound, sulfuric acid compound, nitric acid compound or the like which may be derived from at least one kind of acidic substance selected from the group consisting of hydrochloric acid, sulfuric acid and nitric acid.

Furthermore, the present invention also provides a method for separating a target substance by the use of the above-mentioned particle. The separating method is a method for separating a target substance from a sample by the use of the surface-roughened particles of the present invention, the method comprising the steps of:

(i) bringing the particles of the present invention and the sample containing the target substance into contact with each other, and thereby binding the particles and the target substance to each other;

(ii) allowing the sample to stand, and thereby allowing a spontaneous sedimentation of the particles in the sample; and

(iii) recovering the particles which have precipitated in the sample, and thereby separating the target substance from the sample or obtaining the particles with the target substance immobilized thereon.

The method of the present invention is characterized in that the particles having the target substance which has bound thereto are assembled and aggregated by a spontaneous sedimentation thereof. In other words, the method of the present invention does not make use of a magnetic field or magnetism for a movement and aggregation of the particles. Namely, the separation of the target substance can be achieved only by a spontaneous sedimentation of the particles. This is due to a higher spontaneous sedimentation rate of the particles as compared with that of the prior art.

The particle used in the present method is the surface-roughened particle, and thereby it has more “substance or functional group capable of binding to a target substance” immobilized on the surface thereof. Accordingly, the present method is characterized in that the binding amount of the target substance per particle is larger, so that it is possible to separate a larger amount of target substance from the sample in one procedure, or is possible to obtain a particle with a larger amount of target substance bound thereon in one procedure.

The particles of the present invention preferably have a high density of 3.5 g/cm³ to 9.0 g/cm³ and thus can achieve a sufficient separation rate due to the movement rate of the particles attributable to the spontaneous sedimentation thereof, even without a centrifugal separation or a magnetic separation. As used in this description and claims, the phrase “spontaneous sedimentation (natural sedimentation)” means that particles settle out in a liquid by gravitation. As used in this description and claims, the term “separation” means a separation of a target substance from a sample which contains the target substance. Examples of the target substance include nucleic acids, proteins, sugars, lipids, peptides, cells, eumycetes (fungus), bacteria, yeasts, viruses, glycolipids, glycoproteins, complexes, inorganic substances, vectors, low molecular compounds, high molecular compounds, antibodies and antigens. Examples of the sample include body fluids such as urine, blood, serum, plasma, sperm, saliva, sweat, tears, ascitic fluids and amniotic fluids from humans or animals; suspension liquids, extraction liquids, solutions and crushed solutions of organs, hair, skin, mucous membrane, nail, bone, muscle and nervous tissue from humans or animals; suspension liquids, extraction liquids, solutions and crushed solutions of stools; suspension liquids, extraction liquids, solutions and crushed solutions of cultured cells or cultured tissues; suspension liquids, extraction liquids, solutions and crushed solutions of viruses; suspension liquids, extraction liquids, solutions and crushed solutions of fungus bodies; suspension liquids, extraction liquids, solutions and crushed solutions of soil; suspension liquids, extraction liquids, solutions and crushed solutions of plants; suspension liquids, extraction liquids, solutions and crushed solutions of food and processed food; and drainage water. More specifically, term “separation” substantially means that a target substance contained in a sample is allowed to bind to the particles and then the target substance is separated from the sample by allowing the target substance-binding particles to move. The phrase “separation rate” substantially means a rate of the particle movement in the sample wherein the particles have the target substance which has bound thereto. In a case of “spontaneous sedimentation”, the phrase “separation rate” substantially means a sedimentation rate of particles. The high separation rate can reduce the time required for separating the target substance from the sample. In a case where the particles of the present invention are magnetic particles, the separation rate can be additionally increased by applying a magnetic field thereto.

The spontaneous sedimentation of the particles of the present invention can contribute to a satisfactory separation rate. This means that the use of the particles of the present invention enables simplicity of a separation, immobilization, analysis, extraction, purification or reaction of the target substance. Namely, the use of the particles of the present invention can provide a simple system for performing separation, immobilization, analysis, extraction, purification or reaction of the target substance. In addition, the particles of the present invention are effective for miniaturization or chip processing of the system.

The particle of the present invention is the surface-roughened particle and thus it has an increased surface area on which a larger number of “substance or functional group capable of binding to a target substance” are immobilized. Thus, the amount of the target substance capable of binding to a single particle is increased in the particle of the present invention. As a result, the purification or separation of the target substance can be efficiently performed with the particle of the present invention. In other words, a detectable amount as a whole can increase by the use of the particle of the present invention, which will lead to an achievement of an improved detection sensitivity, a simplified measurement or a reduced measurement error. The particle of the present invention has an increased specific surface area due to the roughening treatment, while the “substance or functional group capable of binding to a target substance” is immobilized on the increased surface of the particle. Accordingly, the amount of the “substance or functional group capable of binding to a target substance” immobilized on the surface of the particle is larger than that of the increased nonspecific bindings accompanied by the increased specific surface area. Thus, the nonspecific binding phenomenon which is accompanied by the increased specific surface area is suppressed with respect to the particle of the present invention. In particular, the ratio of the accumulated volume [cm³] of the micropores having radius of not less than 20 nm per unit surface area [cm²] is 1×10⁻⁶ or more [cm³/cm²] with respect to the particle of the invention, and thus there exists a predetermined amount or more of micropores having larger size than that of the “substance or functional group capable of binding to a target substance” and “target substance”. As a result, the particles of the invention makes it possible not only to immobilize a larger amount of the “substance or functional group capable of binding to a target substance” onto the particle body, but also to allow a larger amount of the target substance to bind to the “substance or functional group capable of binding to a target substance” when the particle is in use (i.e. upon treatment such as separation, immobilization, analysis, extraction, purification or reaction of the target substance). From another viewpoint, the particle of the present invention can achieve a reduced amount of the “smaller-sized micropores (specifically, the micropores having radius of less than 20 nm)” which do not contribute to the immobilization of the “substance or functional group capable of binding to a target substance” or binding of the “target substance”, but adversely contribute to the increase of the nonspecific binding. This means that the particles of the present can increase the binding amount of the target substance per one particle, while suppressing the nonspecific binding phenomenon to a certain extent.

On the body surface of the particle of the present invention, a polymer may be present. In this case, the “substance or functional group capable of binding to a target substance” can be immobilized on the surface of the polymer (hereinafter also referred to as “coating polymer”). The use of the coating polymer makes it possible to immobilize the “substance or functional group capable of binding to a target substance” on the surface of the particle even when it is difficult for the “substance or functional group capable of binding to a target substance” to covalently bond with the particle body. In an application where the immobilized “substance or functional group capable of binding to a target substance” tends to separate from the surface of the particle body due to various conditions, such separation can be prevented by immobilizing the “substance or functional group capable of binding to a target substance” on the coating polymer. In a case where, as the coating polymer, a polymer serving to prevent a penetration of various molecules or metal ions therethrough is selected, an elution of ions from the surface of the particle body or from the inside of the particles can be suppressed (namely, metal ions generated from a constituent component of particles is prevented from being eluted). In this case, an unnecessary reactions caused by the metal ions can be also suppressed in various applications of the particles.

### BRIEF EXPLANATION OF THE DRAWINGS

FIGS. 1(a) to 1(c) schematically illustrate the steps of a method for treating target substance according to the present invention.

FIG. 2 is a micrograph showing particle p1 in Example 1 wherein FIG. 2(a) shows a whole particle p1 and FIG. 2(b) shows an enlarged surface of the particle p1.

FIG. 3 is a micrograph showing particle in Comparative Example 1 wherein FIG. 3(a) shows a whole particle and FIG. 3(b) shows an enlarged surface of the particle.

FIG. 4(a) shows a cross-section of the precursor particle p1 (Example 1) at the vicinity of the surface thereof.

FIG. 4(b) shows an enlarged surface section of the precursor particle of FIG. 4(a).

FIG. 5(a) shows a cross-section of the sulfuric acid-treated particle (Example 1) at the vicinity of the surface thereof.

FIG. 5(b) shows an enlarged surface section of the particle of FIG. 5(a).

FIG. 6(a) shows a cross-section of the porous zirconia particle at the vicinity of the surface thereof.

FIG. 6(b) shows an enlarged surface section of the particle of FIG. 6(a).

FIG. 7 shows graphs showing the relationship between the micorpore radii and “accumulated micropore volume obtained by integrating the volumes of the micropores having the micropore radius of no more than 100 nm from 100 nm side” (Examples 1, 4 and Comparative Examples 1, 2).

FIG. 8 shows enlarged graphs, corresponding to a portion of FIG. 7 (i.e. a portion surrounded by dotted line in FIG. 7).

FIG. 9 shows graphs showing the relationship between the micropore radii of the particles and the micropore volume occupied by each micropore radius (Example 1 and Comparative Example 1).

FIG. 10 shows graphs showing the relationship between the pore radii of the particles and the micropore volume occupied by each pore radius (Example 4 and Comparative Example 2)

In the figures, reference numerals mean the following elements:
1 . . . Particle(s) of the present invention
2″ . . . Target substance(s)
3 . . . Substance(s) other than the target substance(s)
4 . . . Sample

### BEST MODES FOR CARRYING OUT THE INVENTION

First, particles of the present invention will be described, and then a production method of the present invention as well as a separation method of the present invention will be described.

The particles of the present invention have a density suited for separation of a target substance. That is, the particles of the present invention have a density enabling a comparatively high sedimentation rate of the particles when the particles are dispersed in samples, for example, body fluids such as urine, blood, serum, plasma, sperm, saliva, sweat, tears, ascitic fluid and amniotic liquid of humans or animals; suspension liquids, extraction liquids, solutions or crushed solutions of organs, hair, skin, mucous membrane, nail, bone, muscle or nervous tissue of humans or animals; suspension liquids, extraction liquids, solutions or crushed solutions of stools; suspensions liquid, extraction liquids, solutions or crushed solution of cultured cells or cultured tissues; suspension liquids, extraction liquids, solutions or crushed solutions of virus; suspension liquids, extraction liquids, solutions or crushed solutions of fungus bodies; suspension liquids, extraction liquids, solutions or crushed solutions of soil; suspension liquids, extraction liquids, solutions or crushed solutions of plants; suspension liquids, extraction liquids, solutions, or crushed solutions of food and processed food; or drainage water. When the density of the particles is less than 3.5 g/cm³, only the spontaneous sedimentation of the particles will not bring about a preferable movement rate thereof from a practical standpoint. In contrast, the particle density of more than 9.0 g/cm³ is not preferred for a stirring operation upon binding of the target substance. In this regard, the density of the particles of the present invention is in the range of 3.5 g/cm³ to 9g/cm³, preferably in the range of 5.0 g/cm³ to 9.0 g/cm³, and more preferably in the range of 5.5 g/cm³ to 7.0 g/cm³. In some situations, there may be the cases where the present particle has a density larger than 9.0 g/cm³, specifically, a density ranging from 9.0 g/cm³ (except for 9.0 g/cm³) to 23 g/cm³. As used in this description and claims, the term “density” means a true density (real density) in which only a volume occupied by the substances is used as a volume for calculation of density, and such density can be determined by a true density measuring device ULTRAPICNOMETER 1000 (manufactured by Yuasa Ionics Inc.).

With respect to the particle of the present invention, a particle body has a roughened surface so that a specific surface area of the particle is 1.4 to 100 times the specific surface area of a true spherical particle which has the same particle size and the same density as those of the present particle. This means the following matters:
Each of the present particles having the particle size “a” has the specific surface area which is 1.4 to 100 times the specific surface area of a true spherical particle having the particle size “a” (and also having the same density as that of the present particle).
An average value of the specific surface areas of a plurality of the present particles (i.e. particles having the form of powder) having the average particle size “a” is 1.4 to 100 times the specific surface area of a true spherical particle having the particle size “a” (and also having the same density as that of the present particle).
   As will be described later relating to the production process of the present invention, the particle of the present invention is characterized in that the body of the particle has a roughened surface due to a roughening-treatment using at least one kind of acidic substance selected from the group consisting of hydrochloric acid, sulfuric acid and nitric acid (except for phosphoric acid). More specifically, it is preferred that the particle of the present invention is a roughened particle whose body has been roughened by contacting a raw particle (i.e. precursor particle) with the above acidic substance. Due to the surface-roughening treatment, the particle of the present invention has the specific surface area which is 1.4 to 100 times the specific surface area of a true spherical particle having the same particle size and the same density as those of the present particle. In other words, given that the value of the specific surface area of the present particle is expressed by “SP particle” (m²/g) and that the value of the specific surface area of the true spherical particle having the same particle size and the same density as those of the present particle is expressed by “SP true sphere” (m²/g), they have the following relationship:

“SP particle”=1.4×“SP true sphere” to 100×“SP true sphere” (i.e. 1.4×“SP true sphere”≦“SP particle”≦100×“SP true sphere”)

In a case of “SP particle”<1.4×“SP true sphere” (i.e. “SP particle” being less than 1.4×“SP true sphere”), the binding amount of the target substance per one particle will decrease, which leads to a decrease in the detected amount of the target substance bound to the particles as a whole. On the other hand, in a case of “SP particle”>100×“SP true sphere” (i.e. “SP particle” being larger than 100×“SP true sphere”), it is practically undesirable since not only the “nonspecific binding” where the substance other than the target substance binds to the particle body is more likely to occur beyond necessity, but also the particle body becomes brittle in terms of its structure. Preferably, the relationship “SP particle”=1.5×“SP true sphere” to 80×“SP true sphere” stands (i.e. “SP particle” is in the range of 1.5×“SP true sphere” to 80×“SP true sphere”), and more preferably the relationship “SP particle”=1.6×“SP true sphere” to 60×“SP true sphere” stands (i.e. “SP particle” is in the range of 1.6×“SP true sphere” to 60×“SP true sphere”). There are some cases where the relationship “SP particle”=1.4×“SP true sphere” to 500×“SP true sphere” stands (i.e. “SP particle” is in the range of 1.4×“SP true sphere” to 500×“SP true sphere”), depending on the various production conditions such as the conditions of surface roughening treatment and the kind of the material of the precursor particle.

The term “specific surface area” as used in this description and claims corresponds to a specific surface area determined by a specific surface area micropore distribution analyzer BELSORP-mini (manufactured by Bel Japan Inc.). As used in this description and claims, the term “particle size” of the particle of the present invention or the term “particle size” of “true spherical particle having the same particle size and the same density as the particle of the present invention” substantially means the diameter of a true circle having the same area as that of particle image (if the particle body is provided with a polymer coating, “particle image” contains the thickness of the polymer coating). The term “average particle size” substantially means a particle size calculated as a number average by measuring each size of 300 particles for example, based on an electron micrograph or optical micrograph of the particles.

As described above, the particle of the present invention has a roughened surface at the particle body wherein a ratio value of an accumulated volume [cm³] of the micropores having radius of not less than 20 nm per unit surface area [cm²] is not less than 1×10⁻⁶ [cm³/cm²]. The above accumulated micropore volume reflects a micropore size distribution of the surface-roughened particle, and thus meaning that the particle of the present invention has a predetermined amount or more of 20 nm or more-radius sized micropores.

In the particle of the present invention, it is significant that there are micropores with the desired size (i.e. micropore radius of 20 nm or more) in a predetermined amount or more. This is now explained in detail. In a case where the micropore size of the particle is too small, the “substance or functional group capable of binding to a target substance” and “target substance” cannot enter the micropore, which not only causes no contribution of the increased surface area resulted from the micropores to the immobilization of the “substance or functional group capable of binding to a target substance”, but also causes a failure of the target substance to enter the micropores in use of the particle, and thereby inhibiting the access of the target substance to the “substance or functional group capable of binding to a target substance”, if any, located within the micropores. In contrast, when the particle has the micropore size of more than the predetermined level, the “substance or functional group capable of binding to a target substance” as well as the “target substance” can enter such micropores, which not only provides a contribution of the increased surface area resulted from the micropores to the immobilization of the “substance or functional group capable of binding to a target substance”, but also allows the target substance to enter the micropores in use of the particle, and thereby achieving the access of the target substance to the “substance or functional group capable of binding to a target substance” located within the micropores. In other words, in a case where the micropore size of the particle is smaller than the sizes of the “substance or functional group capable of binding to a target substance” and “target substance”, the particle has a large specific surface area in fact, but may serve as if it has substantially no micropore from the viewpoint of the immobilization upon preparation of the particle as well as the viewpoint of the binding of the target substance upon use of the particle. However, as for the particle of the present invention, the ratio of such smaller-sized micropores is reduced, and instead the ratio of the desirable-sized micropores which can contribute to the immobilization of the “substance or functional group capable of binding to a target substance” as well as the binding of the “target substance” is increased. It should be noted that the present invention has been created from the viewpoint of the accumulated volume of micropores having a certain size and more, not from the viewpoint of the accumulated volume of micropores having a whole range of sizes.

In the present invention, “ratio of the desirable-sized micropores” is evaluated on the basis of the unit surface area of the particles or the particle bodies so as to avoid depending on the sizes of the particles. Specifically, it is evaluated by the ratio of “accumulated micropore volume obtained as the sum of the micropore volumes that satisfy the desirable sizes” with respect to unit surface area of the particle (or with respect to unit surface area of the particle body). Such “accumulated micropore volume (i.e. the accumulated volume (cm³/g) obtained as the sum of the volumes of the desirable-sized micropores)” per unit surface area of the particle can be calculated by dividing “accumulated volume of the micropores satisfying the predetermined size condition per gram of the particles or particle bodies (cm³/g)” by “surface area per gram of a true spherical particle having smooth a surface and also having the same particle size and the same density as those of the present particle or particle body (cm²/g)”.

In the particle of the present invention, the accumulated micropore volume regarding the micropores with radius of not less than 20 nm has a certain proportion exceeding a certain value. Specifically, the ratio of the accumulated volume [cm³] of the micropores having radiuses of not less than 20 nm with respect to unit surface area [cm²] is 1×10⁻⁶ [cm³/cm²] and more. As a result, the present invention provides not only an effect that the micropores of the particle can be effectively available for the immobilization of “substances or the functional groups capable of binding to the target substance” and for the binding of the target substance to the “substance or functional group capable of binding to a target substance”, but also an effect that the influence of the nonspecific bindings can be relatively suppressed. When the ratio of the accumulated volume of the micropores having radiuses of not less than 20 nm [cm³] with respect to unit surface area [cm²] is 1×10⁻⁶ [cm³/cm²] and more, the particle can also suppress an apparent nonspecific adsorption in which the trapped target substance within the micropores cannot escape therefrom. As a result, the amount of the nonspecifically-adsorbed substances is reduced as a whole, so that the present particle can be used in a practical use more easily than the prior-art particle. In other words, in a case where the ratio of the accumulated volume [cm³] of the micropores having radiuses of not less than 20 nm with respect to unit surface area [cm²] is less than 1×10⁻⁶ [cm³/cm²], it is hard to immobilize a larger amount of the “substance or functional group capable of binding to a target substance” to the body of particle upon preparation of the particle, and also hard to bind the “target substance” to the particle upon the use of the particle. Accordingly, the above ratio of less than 1×10⁻⁶ [cm³/cm²] is not preferred since the influence of the nonspecific bindings becomes greater.

In another viewpoint apart from the above “accumulated volume of the micropores with radius exceeding a certain value”, it is preferred that the present particle has a porosity (the ratio of pores open at particle surfaces) of not more than 90%. The reason for this is that the particle with the porosity of 90% or more can cause the practical problems. For example, a decreased strength of the particle is caused due to the large portion in micropores of the particle, and thereby the particle is likely to break upon use thereof. The porosity of the particle is preferably in the range from about 0.5% to about 70%.

The particle of the present invention may have or may not have a through-pore in the particle body thereof. However, it is preferred that the particle body of the present invention does not have the through-pore therein from the viewpoint of decreasing the nonspecific adsorption phenomenon. In a case where the particle body does not have the through-pore, it is preferred that the ratio of the accumulated volume [cm³] of the micropores having radius of not less than 20 nm with respect to unit surface area [cm²] is not more than about 4.6×10⁻⁴ [cm³/cm²]. Thus, when considered in combination with the above-mentioned conditions, the particle of the present invention preferably has the ratio of the accumulated volume [cm³] of the micropores having radius of not less than 20 nm with respect to unit surface area [cm²] in the range of from 1×10⁻⁶ to 4.6×10⁻⁴ [cm³/cm²], more preferably in the range of from 3×10⁻⁶ to 1.5×10⁻⁴ [cm³/cm²], still preferably in the range of from 5×10⁻⁶ to 6.5×10⁻⁵ [cm³/cm²], for example in the range of from 6×10⁻⁶ to 8×10⁻⁶ [cm³/cm²]. In this case, the effect of decreasing the nonspecific adsorption phenomenon is remarkably expected.

Assuming that a particle has no through-pore, the surface of the particle body is roughened to a limited depth from the surface of the body, preferably roughened to about 2 μm from the surface of the particle body, more preferably roughened to about 1.5 μm from the surface of the particle body, and still more preferably roughened to about 1 μm from the surface of the particle body. In this regard, a ratio of the roughened portion in the particle is preferably not more than 40% of the diameter of the particle body, more preferably not more than 30% of the diameter of the particle body, and still more preferably not more than 20% of the diameter of the particle body. The expression “ . . . roughened to a limited depth from the surface of the particle body” means that the micropores substantially exist to a limited depth from the surface of the particle body, and thus means that the true spherical particle is roughened to a limited depth from the surface of the body if the term “true spherical particle” is used for expression. The expression “ratio of the roughened portion in the particle (%)” substantially means a ratio of the roughened region (i.e. micropore region) when observed the overall particle along a line passing through the center of the particle.

It should be noted that the value of the “accumulated micropore volume” substantially represents a value obtained by BET method and DH method in the context of the present description.

The BET method is a method for measuring a specific surface area on the basis of a multimolecular layer adsorption model in which Langmuir monomolecular layer adsorption theory is applied to a multimolecular layer adsorption phenomenon. Such BET method is a technique comprising measuring an adsorption volume of a gas (e.g. nitrogen gas) when adsorbed to the particle; subsequently applying a BET equation as shown below to the obtained adsorption isotherm, thereby obtaining the value Vm of the monomolecular layer adsorption volume of the gas; and then calculating the specific surface area of the particle by using the obtained value Vm and the molecular cross-sectional area of the adsorbed gas molecule. The detailed explanation of the measurement of the specific surface area according to the BET method is specified by JIS Z8830:2001.

While on the other hand, the DH method is an abbreviation of the Dollimore-Heal method, which is an analytical method for obtaining a volume-frequency distribution of the micropore sizes by using a relative pressure of the adsorption gas and the increments of the adsorption amount thereof, assuming that the each micropore has a cylindrical shape.

$$\frac{P}{V ( {P}_{0} - P )} = \frac{1}{VmC} + ( \frac{C - 1}{VmC} ) ( \frac{P}{{P}_{0}} ) \qquad [ BET equation ]$$

wherein

P is an adsorption equilibrium pressure in an adsorption equilibrium state at a constant temperature;

V is an adsorption amount at an adsorption equilibrium pressure P;

P₀ is a saturated vapor pressure;

Vm is a monolayer adsorption volume (i.e. adsorption volume, given that a gas molecule forms a monolayer on a solid surface); and

C is a BET constant (a parameter relating to heat of adsorption).

More specifically, the value of the “accumulated micropore volume” corresponds to a value calculated according to the DH method based on an adsorption isotherm of the particle wherein the adsorption isotherm is measured until the relative pressure (P/P₀) reaches 0.99 by the use of the specific surface area micropore distribution analyzer Belsorp-mini (manufactured by Bel Japan Inc.).

It is preferred that the particles of the present invention have the particle size or average particle size in the range of 1 μm to 5 mm. When the particle size or average particle size is less than 1 μm, it becomes difficult to sufficiently increase the particle movement rate attributable to spontaneous sedimentation upon separating the target substance. In contrast, when the particle size or average particle size is more than 5 mm, the sedimentation of the particles is completed before the binding of the target substance thereto, which will lead to an unsatisfactory separation of the target substance. The particle size or average particle size is more preferably in the range of 1 μm to 1 mm, still more preferably in the range of 5 μm to 500 μm, and the most preferably in the range of 10 μm to 100 μm. As the size or the average size of the particles becomes smaller, a rapid oxidation may occur, and thereby an ignition of the particles may also occur. In this regard, the comparatively large size or average size of the particles according to the present invention contributes to the prevention of the rapid oxidation and ignition of the particles.

As described above, it is possible to achieve a satisfactory separation rate only by the spontaneous sedimentation of the particles of the present invention. In other words, a spontaneous sedimentation rate of the particles of the present invention is high in a sample containing a target substance.

The material of the particle body is not limited as long as the particles of the present invention have the above-described density and specific surface area. For example, in the case where the particles have the density of 3.5 g/cm³ to 9 g/cm³, it is preferred that the particle body is formed of a metal or metal oxide. More specifically, it is preferred that the particle body is formed of at least one kind of material selected from the group consisting of zirconia (zirconium oxide, yttrium-doped zirconium oxide), iron oxide, alumina, nickel, cobalt, iron, copper and aluminum. In another case where the particles have the density of 9.0 g/cm³ (except for 9.0 g/cm³) to 23 g/cm³, it is preferred that the particle body of the present invention is formed of at least one kind of transition metal element selected from the group consisting of Ag (silver), Au (gold), Pt (platinum), Pd (palladium), W (tungsten), Rh (rhodium), Os (osmium), Re (rhenium), Ir (iridium), Ru (ruthenium), Mo (molybdenum), Hf (hafnium) and Ta (tantalum) or formed of at least one kind of typical metal element selected from the group consisting of Pb (lead), Bi (bismuth) and Tl (thallium).

It is beneficial that the particles of the present invention are magnetized (hereinafter, the magnetized particles of the present invention are referred to as “magnetic particles”), since an auxiliary magnetic separation can be additionally applied to the sedimentation of the particles. When the auxiliary magnetic separation is additionally applied, the particles are allowed to move more quickly, which will lead to a shorter time required for separating a target substance (more specifically a shorter time required for separating “target substance which have bound to the particles”). Further, a pipetting or decantation operation can be easily performed by collecting or settling the particles by means of magnetism.

In a case where the coating polymer is present on the surface of a particle body, it is usually difficult to impart magnetism to the coating polymer. It is therefore preferred that a magnetized particle is used as the particle body.

The material for the bodies of the magnetic particles is not limited as long as the particles are magnetized. For example, it is preferred that the bodies of the magnetic particles are formed of at least one kind of iron oxide selected from the group consisting of a garnet-structured oxide comprising a transition metal and an iron, ferrite, magnetite, and γ-iron oxide. Alternatively, the bodies of the magnetic particles may contain at least one kind of metallic material selected from the group consisting of nickel, cobalt, iron and alloy thereof. As used herein, “garnet-structured oxide comprising a transition metal and an iron” is generally referred to as YIG. For example, “garnet-structured oxide comprising a transition metal and an iron” is a compound represented by the composition formula of Y₃Fe₅O₁₂, or BiₓY₃₋ₓFe₅O₁₂ (0<X<3) in which a portion of Y in the compound is substituted with bismuth.

Alternatively, the magnetic particles may be formed by coating or attaching a magnetic substance to non-magnetized particles. Upon coating or attaching the magnetic substance, an electroless plating process, electroplating process, sputtering process, vacuum deposition process, ion plating process or chemical deposition process can be employed. Examples of “non-magnetized particles” as used herein include high density particles formed of zirconia (zirconium oxide, yttrium-doped zirconium oxide), alumina or the like. When the content of the high-density magnetic substance is higher, lower-density particles formed of aluminum, silica or resin can be also used. Examples of “magnetic substance” used for coating or attaching include iron oxides such as ferrite, magnetite, γ-iron oxide and garnet-structured oxide comprising a transition metal and an iron, which are similar to the above-described material for the magnetic particles. Nickel, cobalt, iron or an alloy thereof may also be used as the magnetic substance.

In the case where the magnetic particles are formed by coating or attaching the magnetic substance on the surface of particles, if the amount of the magnetic substance to be formed on the surfaces of the particles is too small, the intensity of the magnetization of the particles decreases. This is not preferred for magnetic separation. It is therefore preferred that the volume of the coating magnetic substance accounts for 5% or more of the volume of particles (i.e. particles with the coating magnetic substance thereon). As for a thickness of the coating magnetic substance of each particle, it is preferred that such thickness accounts for 1.7% or more of the diameter of each particle (i.e. particle with the coating magnetic substance thereon). It should be noted that not only an embodiment wherein the coating magnetic substance is formed on “non-magnetized particles”, but also an embodiment wherein a magnetic substance is included inside “non-magnetized particles” is possible.

Magnetic characteristics of magnetic particles include, for example, “saturation magnetization” and “coercive force (coercitivity)”. As the value of the saturation magnetization increases, the responsiveness of particles to magnetic field is generally improved. In order to magnetize the particles having a comparatively high density, it is necessary to supply a magnetic substance on the surface of or inside the non-magnetized particles. In this regard, the magnetic substance has a density smaller than that of the non-magnetized particles, and thus the required density must be achieved by restricting the amount of the magnetic substance to be supplied. When the particle body is coated with the non-magnetic polymer, it is actually difficult to achieve a saturation magnetization higher than that of particles formed of only the magnetic substance. In other words, it is actually difficult to achieve a saturation magnetization of more than 85 A·m²/kg. In contrast, when the saturation magnetization is less than 0.5 A·m²/kg, the responsiveness of the particles to the magnetic field falls below a required level and thus it is not preferred. Therefore, the saturation magnetization of the particles of the present invention is preferably in the range of 0.5 A·m²/kg to 85 A·m²/kg (0.5 emu/g to 85 emu/g), more preferably in the range of 3 A·m²/kg to 30 A·m²/kg (3 emu/g to 30 emu/g), for example 4A·m²/kg to 15 A·m²/kg (4 emu/g to 15 emu/g). When the value of the coercive force increases, the particles tend to aggregate. However, when the value of the coercive force is too large, the dispersion of the particles is inhibited due to an excessively strong aggregation action. Namely, too large coercive force is not preferred in terms of the binding of the target substance. Therefore, the coercive force is preferably in the range of 0 kA/m to 23 KA/m (0 to 300 Oe), more preferably in the range of 0 kA/m to 15.95 kA/m (0 to 200 Oe), and still more preferably in the range of 0 kA/m to 7.97 kA/m (0 to 100 Oe).

The values of “saturation magnetization” and “coercive force” as used in this description are values measured by a vibration sample type magnetometer (manufactured by TOEI INDUSTRY CO., LTD., Model VSM-5). Specifically, the value of “saturation magnetization” is a value determined from the magnetization amount when the magnetic field of 797 kA/m (10 kOe) is applied. The value of “coercive force” is a value of the applied magnetic field at which the magnetization amount becomes zero when the magnetic field is returned to zero after applying the magnetic field of 797 kA/m, and then the magnetic field is gradually increased in the reverse direction.

There is no restriction on the shape of particles of the present invention. Each shape of the particles may be sphere, ellipsoid, granule, plate, needle or polyhedron (e.g. cube). In order to decrease variation between particles in terms of the binding of the target substance thereto, each shape of the particles is preferably a regular shape, and more preferably a spherical shape. In a case where the coating magnetic substance is provided on the body of the non-magnetized particle, it is preferred that “body of non-magnetized particle” has a spherical or ellipsoidal shape.

It is preferred that “substance capable of binding to a target substance” (hereinafter also referred to as “substance to which a target substance can bind”) immobilized on the body surface of each particle of the present invention is at least one kind of a substance selected from the group consisting of biotin, avidin, streptavidin and neutravidin. It is preferred that “functional group capable of binding to the target substance” (hereinafter also referred to as “functional group to which a target substance can bind”) immobilized on the body surface of each particle of the present invention is at least one kind of a functional group selected from the group consisting of carboxyl group, hydroxyl group, epoxy group, tosyl group, succinimide group, maleimide group, thiol group, thioether group, sulfide functional group (e.g. disulfide group), aldehyde group, azido group, hydrazide group, primary amino group, secondary amino group, tertiary amino group, imide ester group, carbodiimide group, isocyanate group, iodoacetyl group, halogen-substitution of carboxyl group and double bond. “Functional group to which a target substance can bind” may be derivatives of these functional groups.

As used in this description and claims, the term “immobilization (immobilized)” substantially means an embodiment wherein “substance to which a target substance can bind” or “functional group to which a target substance can bind” exists in the vicinity of the surface of each particle body. Namely, the term “immobilization (immobilized)” does not necessarily mean only the embodiment wherein “substance to which a target substance can bind” or “functional group to which a target substance can bind” is directly attached to the surface of each particle body. Also, the term “immobilization (immobilized)” substantially means an embodiment wherein “substance or functional group to which a target substance can bind” is immobilized on at least a part of each particle surface. Accordingly, “substance or functional group to which a target substance can bind” is not necessarily immobilized over the entire surface of each particle. In a preferred embodiment, “substance or functional group to which a target substance can bind” is present on the entire surface of each particle so that each particle body is surrounded by “substance or functional group to which a target substance can bind”. As used in this description and claims, the expression “target substance binds” includes not only an embodiment wherein a target substance is “adsorbed” or “absorbed” to particles, but also an embodiment wherein a target substance binds to particles due to various kinds of “affinities” acting between the target substance and the particles.

According to the present invention, due to the fact that “substance to which a target substance can bind” or “functional group to which a target substance can bind” is immobilized on the body of each particle, the target substance can bind to the particle via such substance or functional group.

In a preferred embodiment, a coating or adhering polymer is provided on a part of the surface of the particle body or the whole surface of the particle body, and “substance or functional group capable of binding to a target substance” is immobilized on the surface of the particle body and/or the polymer. In another preferred embodiment, a coating or adhering polymer is provided on the entire surface of the particle body, and “substance or functional group capable of binding to a target substance” is immobilized on the surface of the polymer. As the coating or adhering polymer to be provided on the surface of the particle body, some polymer which contributes to the immobilization of “substance to which a target substance can bind” or “functional group to which a target substance can bind” is preferred. In this case, the kind of the coating polymer or adhering polymer can be selected on the basis of the kind of “substance to which a target substance can bind” or “functional group to which a target substance can bind”, conditions of use for particles, or other required characteristics of the particles. The representative examples of the coating polymer include at least one kind of synthetic polymer compound selected from the group consisting of polystyrene or derivatives thereof, poly(meth)acrylic acid, poly(meth)acrylic acid ester, polyvinylether, polyurethane, polyamide, polyvinyl acetate, polyvinyl alcohol, polyallylamine, and polyethyleneimine. The polymer is not limited to such synthetic polymer compound and may be a modified polymer or a copolymer thereof. Furthermore, for example, a semi-synthetic polymer compound such as hydroxyalkyl cellulose, carboxyalkyl cellulose and sodium alginate; or a natural polymer compound such as chitosan, chitin, starch, gelatin and gum arabic may be used. Still furthermore, a polymer having a functional group introduced thereto in advance may be used, wherein “substance or functional group capable of binding to a target substance” can bind and adhere to such functional group.

In a case where the main objective is to suppress an elution of metal ions (namely, ions of metal constituting a particle body) from the surface or inside of particles, the coating polymer capable of hindering the penetration of the various molecules or metal ions constituting the particle body may be used. When the particles is intended for the use in an aqueous system, the coating polymer capable of hindering a penetration of water may be used, and in this case, polystyrene, alkyl polymethacrylate, polyvinylether or polyvinyl acetate can be used, for example.

In this description or claims, “coat”, “attach” or “adhere” substantially means an embodiment where a polymer exists on at least a part of the surface of the particle.

Hereinafter, the binding of the particles with the target substance will be described in detail. When the particles of the present invention and the target substance are allowed to coexist, the target substance can bind the particles due to an adsorptivity or affinity generated between “substance or functional group capable of binding to a target substance” of the particle and the target substance. In the classification below, “adsorption” is defined to have the same meaning as “chemical adsorption”.

As an example of embodiment wherein a target substance binds to particles due to the adsorptivity, “target substance” is avidin, a particle body is made of zirconia, and “substance or functional group capable of binding to a target substance” is an epoxy group.

With respect to “affinity”, “substance or functional group capable of binding to a target substance” immobilized on the surface of the particle body can be roughly classified into the following five kinds, based on the kind of the affinity generated between “substance or functional group capable of binding to a target substance” and the target substance (it should be noted that substances or functional groups exemplified in each classification are only for illustrative purposes and other substances or functional groups are also possible). When involved in the affinity as described above, “substance or functional group capable of binding to a target substance” is hereinafter referred to also as “substance or functional group having affinity”.

(1) Examples of “substance or functional group having affinity with a target substance” wherein the affinity results from electrostatic interaction, π-π interaction, π-cation interaction, or dipole-dipole interaction:

Silica, activated carbon, sulfonic acid group, carboxyl group, diethylaminoethyl group, triethylaminoethyl group, phenyl group, arginine, cellulose, lysin, polylysin, polyamide, poly(N-isopropylacrylamide), crown ether or cyclic compound having π electrons, and functional group derivatives, oxygen conjugates and fluorescence probe conjugates thereof.

(2) Examples of “substance or functional group having affinity with a target substance” wherein the affinity results from hydrophobic interaction:

Alkyl group, octadecyl group, octyl group, cyanopropyl group, butyl group, phenyl group, and functional group derivatives, oxygen conjugates and fluorescence probe conjugates thereof.

(3) Examples of “substance or functional group having affinity with a target substance” wherein the affinity results from hydrogen bond:

DNA, RNA, Oligo (dT), chitin, chitosan, amylose, cellulose, dextrin, dextran, pullulan, polysaccharide, lysin, polylysin, polyamide, poly(N-isopropylacrylamide), β-glucan, and functional group derivatives, oxygen conjugates and fluorescence probe conjugates thereof.

(4) Examples of “substance or functional group having affinity with a target substance” wherein the affinity results from coordinate bond:

Iminodiacetic acid, nickel, nickel ion, nickel complex, cobalt, cobalt ion, cobalt complex, copper, copper ion and copper complex, and oxygen conjugates and fluorescence probe conjugates thereof.

(5) Examples of “substance or functional group having affinity with a target substance” wherein the affinity results from a biochemical interaction (biochemical interaction means an interaction including an interaction relating to biological molecules, such as antigen-antibody reaction, ligand-receptor bond, hydrogen bond, coordinate bond, hydrophobic interaction, electrostatic interaction, π-π interaction, π-cation interaction, dipole-dipole interaction and van der Waals force acting alone or in combination thereof):

Antigen, antibody, receptor, ligand, biotin, avidin, streptavidin, Neutravidin, silica, activated carbon, magnesium silicate, hydroxyapatite, albumin, amylose, cellulose, lectin, protein A, protein G, S protein, dextrin, dextran, pullulan, polysaccharide, calmodulin, nickel, nickel ion, nickel complex, cobalt, cobalt ion, cobalt complex, copper, copper ion, copper complex, gelatin, N-acetylglucosamine, iminodiacetic acid, aminophenylboric acid, ethylenediaminediacetic acid, aminobenzamidine, arginine, lysin, polylysin, polyamide, diethylaminoethyl group, triethylaminoethyl group, ECTEOLA-cellulose, fibronectin, vitronectin, peptides containing an arginine-glycine-aspartic (RGD) acid sequence, laminin, poly(N-isopropylacrylamide), collagen, concanavalin A, adenosine5′ phosphoric acid (ATP), ADP, ATP, nicotinamide adenine dinucleotide, acridine dye, aprotinin, ovomucoid, inhibitors (e.g. trypsin inhibitor and protease inhibitor), phosphorylethanolamine, phenylalanine, protamine, cibacron blue, Procion Red, heparin, glutathione, DIG, DIG antibody, DNA, RNA, Oligo (dT), chitin, chitosan, β-glucan, calcium phosphate, calcium hydrogenphosphate, hyaluronic acid, elastin, sericin and fibroin, and functional group derivatives, oxygen conjugates and fluorescence probe conjugates thereof.

As is apparent from the above classification, the expression “having affinity” as used herein substantially means that an electrostatic interaction, a π-π interaction, a π-cation interaction, a dipole-dipole interaction, a hydrophobic interaction, a biochemical interaction, a hydrogen bond or a coordinate bond is generated between a target substance and a substance or functional group immobilized on the particles. It should be noted that the substance or functional group may have two or more kinds of affinities according to the kind of the substance or functional group to be immobilized on the particle body and there may be overlapping substance or functional group in the above classification. There is no restriction on the above classification, and any suitable substances or functional groups may be immobilized on the particles as long as it has a function of acting on a target substance so as to allow the target substance to exist on the surfaces of particles or in the vicinity thereof. For example, substances or functional groups having affinity due to a complementary shape with a target substance may be immobilized.

Hereinafter, the method of producing the particle of the present invention will be described in detail. The method of producing the particle of the present invention is a method of producing a particle to which a target substance can bind, the surface of which particle is a roughened surface with a specific surface area being 1.4 to 100 times the specific surface area of a true spherical particle having the same particle size and the same density as the present particle.

The method of the present invention comprises the steps of:

(I) contacting precursor particles (i.e. raw particles) with at least one kind of acidic substance selected from the group consisting of hydrochloric acid, sulfuric acid and nitric acid (except for phosphoric acid); and

(II) immobilizing “substance or functional group capable of binding to a target substance” to the precursor particles.

In the step (I), the precursor particles are brought into contact with at least one kind of acidic substance selected from the group consisting of hydrochloric acid, sulfuric acid and nitric acid. When the particles with the density of 3.5 g/cm³ to 9g/cm³ are intended to be produced, it is preferred that the precursor particles are formed of a metal or metal oxide. More specifically, the precursor particles are formed of at least one kind of a material selected from the group consisting of zirconia (zirconium oxide, yttrium-doped zirconium oxide), iron oxide, alumina, nickel, cobalt, iron, copper and aluminum. In this regard, the density of the precursor particles of the present invention is in the range of 3.5 g/cm³ to 9.0 g/cm³, preferably in the range of 5.0 g/cm³ to 9.0 g/cm³, and more preferably in the range of 5.5 g/cm³ to 7.0 g/cm³. In some situations, the precursor particles may have a density more than 9.0 g/cm³, more specifically in the range of 9.0 g/cm³ to 23 g/cm³ (except for 9.0 g/cm³). In such a case, the precursor particles are preferably formed of at least one kind of transition metal element selected from the group consisting of Ag (silver), Au (gold), Pt (platinum), Pd (palladium), W (tungsten), Rh (rhodium), Os (osmium), Re (rhenium), Ir (iridium), Ru (ruthenium), Mo (molybdenum), Hf (hafnium) and Ta (tantalum), or preferably formed of at least one kind of typical metal element selected from the group consisting of Pb (lead), Bi (bismuth) and Tl (thallium). The precursor particle has the particle size or average particle size which is preferably in the range of 1 μm to 5 mm, more preferably in the range of 5 μm to 500 μm and still more preferably in the range of 10 μm to 100 μm. In addition, the precursor particle to be used is preferably a particle with no through-pore. That is, the precursor particle is substantially solid and thus the particle has no “interpenetrating network structure”. Any of commercially available ones, which implements the above material and properties, may be used as the precursor particle.

Preferably, “acidic substance selected from the group consisting of hydrochloric acid, sulfuric acid and nitric acid” is used in a liquid state. Among these acidic substances, it is particularly preferable to use sulfuric acid or nitric acid. Upon contacting the precursor particle with the above acidic substance, it is preferable to subject a mixture containing the precursor particle and the acidic substance to a heat treatment or a hydrothermal reaction (or a solvothermal method). In a case of the hydrothermal reaction (or solvothermal method), each of hydrochloric acid, sulfuric acid and nitric acid means hydrochloric aqueous solution, sulfuric aqueous solution and nitric aqueous solution, respectively, and also the mixture containing the precursor particles and the acidic substance is in a form of an aqueous solution. The acid concentration of the acidic substance may be any suitable ones, depending on kind of acid, temperature, pressure, treating time, handling ability, cost, safety or the like (for example in the case where the reaction is carried out in a pressure tight vessel under a temperature condition of 150° C. to 250° C. for 3 hours to 16 hours, the sulfuric acid may be used in its concentration of 10 vol % to 30 vol %). In the hydrothermal reaction, the mixture containing the precursor particles and the acidic substance is heated up to a proper temperature. For example, an autoclave, a thermostatic bath or a microwave irradiator can be used for the heating. The temperature condition for the hydrothermal reaction is preferably in the range of 150° C. to 300° C., more preferably in the range of 160° C. to 280° C., and still preferably in the range of 170° C. to 240° C. The pressure condition for the hydrothermal reaction is preferably in the range of 0.4 to 10 MPa, more preferably in the range of 0.5 to 7 MPa and still preferably in the range of 0.7 to 3.7 MPa. It is preferred that the hydrothermal reaction is performed for a time period of generally from 1 min to 12 hours, preferably from 30 min to 9 hours, and more preferably from 1 hour to 7 hours. In the solvothermal method, not only water but also various organic solvents can be used. In such a case, the kind of the solvent is not particularly restricted as long as it can avoid forming a two-phase system with the acidic substance being used. In this reaction, the reaction temperature described above is applicable. The pressure condition may vary depending on the kind of the solvent to be used, however it is unambiguously defined when the temperature condition is decided.

For example, in a case where a microwave is used for the hydrothermal reaction, the mixture containing the precursor particles and the acidic substance is charged into a pressure tight vessel, and then the mixture solution is irradiated with the microwave from outside thereof. The irradiation of microwave is continued until the temperature of the mixture containing the precursor particle and the acidic substance reaches the target temperature. Even after reaching the target temperature, the irradiation may be continued while varying power of the microwave so as to keep the temperature constant. The frequency of the microwave to be irradiated is not particularly restricted as long as it can heat the mixture containing the precursor particles and the acidic substance up to the target temperature (i.e. the temperature of from 150° C. to 300° C.), it is 2.45 GHz for example. The power of the microwave to be irradiated is also not particularly restricted as long as it can heat the mixture up to the target temperature. However, when the power is high, the period for being required to reach the target temperature is shortened, on the other hand when the power is low, the temperature of the mixture solution can be easily controlled to keep it constant. It is particularly preferred that the power of the microwave can be variably controlled, since both of the period shortening and the temperature controlling can be suitably performed. As the apparatus capable of variably controlling the power of the microwave, MicroSYNTH (manufactured by Milestone-General Co.) may be used.

In the step (I), the surface of the precursor particle is roughened, so that the particle has a specific surface area which is 1.4 to 100 times the specific surface area of a true spherical particle having the same particle size and the same density as those of the present particle, and preferably the ratio of the accumulated volume [cm³] of micropores having radius of not less than 20 nm per unit surface area [cm²] is not less than 1×10⁻⁶ [cm³/cm²] in the particle or particle body. According to the roughening treatment of the step (I), the precursor particle may be surface-roughened to the depth of about 2 μm from the surface of the particle, preferably surface-roughened to the depth of about 1.5 μm from the surface of the particle, and more preferably surface-roughened to the depth of about 1 μm from the surface of the particle. However, a ratio of the roughened portion in the particle is preferably not more than 40% of the diameter of the particle body, more preferably not more than 30% of the diameter of the particle body, and still more preferably not more than 20% of the diameter of the particle body. The expression “ratio of the roughened portion in the particle (%)” substantially means a ratio of the roughened region (i.e. micropore region) when observed the overall particle along a line passing through the center of the particle.

Subsequent to the step (I), it is preferable to subject the precursor particles to a washing treatment, filtering treatment or drying treatment. The washing treatment of the particles can remove impurities from the surface of each of the particles. In particular, the acidic substance used in the surface roughening treatment or the compound derived therefrom can be removed. The washing treatment of the particles is preferably performed by rising them with water. However, any suitable liquid other than the water may also be used. For example, alcoholic solvents such as ethanol, methanol and various organic solvents such as toluene and hexane can be used for the washing treatment of the particles. The filtration treatment can be performed together with the washing treatment in order to remove the washing solution from the washed particles. It is preferable to perform the drying treatment of the particles under a temperature condition of 10 to 150° C., more preferably of 40 to 90° C. The drying treatment may be performed with a dryer, but it can be nevertheless performed by a natural drying.

Then, in the step (II), the “substance or functional group capable of binding to a target substance” is immobilized to the precursor particles. That is, the “substance to which a target substance can bind” or the “functional group to which a target substance can bind” is introduced into the surfaces of the precursor particles. The technique for immobilizing the “substance to which a target substance can bind” to the surfaces of the precursor particles is not particularly restricted, and any suitable techniques may be applied as long as they allow the “substance to which a target substance can bind” to bind or adhere to the precursor particles. It is not necessarily the case that “substance to which a target substance can bind” directly binds or adheres to the precursor particles. If necessary, the immobilization of “substance to which a target substance can bind” on the precursor particles may be facilitated by introducing other substances, for example, a silicon-containing substance (e.g. siloxane, silane coupling agent and sodium silicate) or a resin having a functional group to which a target substance can bind or adhere, to the bodies of the precursor particles in advance. Alternatively, a noble metal may be provided on the surfaces of the precursor particles, followed by the attaching or introducing of other substances such as a sulfur-containing compound having a functional group to which a target substance can bind or adhere. In a case where the silicon-containing substance is used, the silicon-containing substance and the immobilized “substance to which a target substance can bind” are present on the surfaces of the bodies of the precursor particles.

Just as an example, a silane coupling agent having an epoxy group or an amino group may be introduced to the surfaces of the precursor particle bodies through a reaction so as to immobilize “substance to which a target substance can bind” on the surfaces of the precursor particle bodies.

As with the immobilization of “substance to which a target substance can bind”, there is no restriction on the technique for immobilizing “functional group to which a target substance can bind” on the precursor particles. That is to say, any suitable techniques may be used as long as they allow “functional group to which a target substance can bind” to bind or adhere to the bodies of the precursor particles. If necessary, “functional group to which a target substance can bind” may be converted into another functional group by a chemical treatment, and thereby its reactivity or adsorptivity is changed. As with the case of “substance to which a target substance can bind”, it is not necessarily the case that “functional group to which a target substance can bind” directly binds or adheres to the bodies of the precursor particles. If necessary, the immobilization of “functional group to which a target substance can bind” on the precursor particles may be facilitated by introducing other substances, for example, a silicon-containing substance (e.g. siloxane, silane coupling agent and sodium silicate) or a resin having a functional group to which a target substance can bind or adhere, to the bodies of the precursor particles in advance. Alternatively, a noble metal may be provided on the surfaces of the precursor particles, followed by the attaching or introducing other substances such as a sulfur-containing compound having a functional group to which a target substance can bind or adhere. In a case where a silicon-containing substance is used, the silicon-containing substance and the immobilized “functional group to which a target substance can bind” are present on the surfaces of the bodies of the precursor particles.

Hereinafter, a technique by the use of a silane coupling agent will be described as an example of the technique for immobilizing “functional group to which a target substance can bind” on the particles.

### <<Immobilization of Functional Group by the Use of Silane Coupling Agent>>

This technique is a technique to coat the surfaces of the precursor particles with a silane coupling agent such as 3-glycidoxypropylmethyldiethoxysilane. Such technique has an advantage that the kinds of the functional group can be easily changed by the use of the silane coupling agent in which the terminal-functional group has been modified.

Precursor particles, which were subjected to a surface roughening treatment, are dispersed in pure water and 3-glycidoxypropylmethyldiethoxysilane having a terminal epoxy group is added to the resultant dispersion while stirring. In this case, 3-glycidoxypropylmethyldiethoxysilane may be added solely or in combination with a solvent such as water, ethanol and the like for the purpose of dilution. The ratio of water and the organic solvent can suitably vary. As a catalyst for the reaction, an acid (e.g. acetic acid and hydrochloric acid) or a base (e.g. aqueous ammonia) may be added. The reaction time is usually in the range of from 10 min to 6 hours. When the reaction time is too short, the reaction hardly proceeds. When the reaction time is too long, the epoxy group is likely to decompose. The stirring process is not particularly limited, and a stirring blade, a magnetic stirrer, a disc rotor and the like may be used.

Subsequently the drying step is performed. In this step, it is possible to perform drying not only after rinsing the particles with water, but also after rinsing the particles with an organic solvent. As such organic solvent, various solvents including acetone, toluene and the like may be used. The drying process is not particularly limited, and thus the drying may be performed at a room temperature under a reduced pressure or a normal pressure.

In this way, particles made of yttrium-doped zirconium oxide with an epoxy group immobilized thereon can be finally obtained.

Next, one preferred case where a polymer is used for attaching it to or coating it on the particles will be described. The technique for providing the coating or adhering polymer on the particles is not particularly limited, and any suitable techniques may be applied as long as they make it possible to provide a polymer on at least a part of the surface of each of the particles. For example, the following techniques may be used:
(1) A technique of initiating polymerization from the surfaces of the precursor particles;
(2) A technique of depositing a polymer on the surfaces of the precursor particles by performing a polymerization under the presence of the precursor particles;
(3) A technique of polymerizing through enclosing the precursor particles in a monomer emulsion; and
(4) A technique of mixing a solution of a preliminarily polymerized polymer with precursor particles, and thereby depositing the polymer on the surfaces of the precursor particles.

The above techniques will be described in more detail. With respect to the technique (1), the coating polymer is provided on the surfaces of the precursor particles by binding or adsorbing an initiator and a chain transfer agent on the surfaces of precursor particles, followed by extending the polymer from the surfaces of the precursor particles. With respect to the technique (2), the coating polymer is provided on the surfaces of the precursor particles by performing a polymerization under the presence of precursor particles by the use of a monomer capable of depositing as the polymerization reaction proceeds. Such provision of the polymer can be efficiently performed by selecting electric charges of the polymer and the particles so as to attract them to each other or by immobilizing a polymerizable double bond on the surfaces of the particles. With respect to the technique (3), a combination of a solvent and a monomer capable of forming a monomer emulsion therefrom is selected and precursor particles are included within such monomer emulsion. To this end, a polymerization is carried out so as to provide the coating polymer on the surfaces of the precursor particles. In this technique, a surface treatment or surfactant for improving affinity with the monomer may be used so that the precursor particles preferentially exist in the monomer emulsion. With respect to the technique (4), the coating polymer is provided on the surfaces of the precursor particles by incorporating the precursor particles into a polymer solution, followed by decreasing the solubility of the polymer and thus depositing the polymer through adding a poor solvent, varying the pH or adding a large amount of a salt. In this technique, the provision of the polymer can be efficiently performed by selecting electric charges of the polymer and the precursor particles so as to attract them to each other or by immobilizing a polymerizable double bond on the surfaces of the precursor particles.

Also, the precursor particles may be alternately immersed in polymer solutions each having different electric charge to form a lamination layer(s) on the surfaces of the particles.

In the above-described techniques, various processes such as a microencapsulation and an emulsion polymerization, which have conventionally been known, are available.

Prior to the provision of the coating polymer, the surfaces of precursor particles may be subjected to a particular treatment. Examples of such treatments include a magnetization treatment, a coating treatment with a metal or an inorganic substance, an adsorption treatment with a surfactant, a treatment with a reactive substance such as a silane coupling agent or a titanium coupling agent, a siloxane coating treatment, a treatment for introducing a functional group to Si—H of siloxane (hydrosilylation reaction), an acid treatment or alkali treatment, a solvent washing treatment, a polishing treatment and the like. These treatments contribute to a removal of stains from the surfaces of precursor particles, a control of electric charge for the surfaces of the precursor particles, or an introduction of a reactive functional group to the surfaces of the particles, which will lead to an improvement of the provision of the coating polymer or the adhesion between the coating polymer and the surfaces of the particles. In a case where the silicon-containing substance (e.g. siloxane or silane coupling agent) is used, it should be understood that, in addition to “substance or functional group to which a target substance can bind” and the coating polymer, such silicon-containing substance exists on the body surfaces of the particles of the present invention. For example, the silicone-containing compound may exist between the surface of the particle body and the surface of the coating polymer. By preliminarily attaching or adsorbing an initiator and/or a polymerizable double bond onto the surfaces of the precursor particles, the polymer is likely to deposit on the surfaces of the particles upon polymerization. This is effective for providing the coating polymer on the surfaces of the particles. In addition, it is possible to employ other processes to give other effects such as a reduction effect of nonspecific binding, a suppression effect of elution of metal ions, an adjustment effect of density and an imparted effect of color and fluorescence.

The coating polymer may be subjected to a crosslinking treatment. When the coating polymer is crosslinked, characteristics such as durability, solvent resistance and low swelling of the coating polymer can be improved. There is no restriction on a technique for forming the crosslinked polymer. The typical techniques are classified as follows:

(1) a. Crosslinking upon polymer-coating treatment of precursor particles,
b. Crosslinking after polymer-coating treatment of precursor particles

(2) a. Addition of a crosslinking agent (including crosslinking reaction which proceeds at room temperature or low temperature),
b. Introduction of a crosslinkable functional group into polymer

(3) a. Thermocrosslinking,
b. Radiation crosslinking

It should be noted that the above techniques (1), (2) and (3) can be used in combination. Examples of the combination of “(1) a”, “(2) a” and “(3) a” include a technique wherein a heat treatment is performed with a bifunctional monomer upon providing a coating polymer by initiating polymerization from the surfaces of precursor particles or depositing the polymer on the surfaces of the precursor particles, and a technique wherein a heat treatment is performed with a bifunctional monomer upon polymerizing by including the precursor particles in a monomer emulsion. With respect to the combination of “(1) b”, “(2) a” and “(3) a”, for example, a polyfunctional epoxy crosslinking agent is added and then a heating treatment for crosslinking is carried out after the coating polymer is provided by a deposition of a polymer having a carboxyl group or by a polymerization of a monomer having a carboxyl group. The same is true for the case wherein a hydroxyl group is used instead of the carboxyl group and an isocyanate crosslinking agent is used instead of the epoxy crosslinking agent. An example of “(2) b” includes a technique wherein an epoxy group, an isocyanate group or a double bond is introduced into a coating polymer. In this case, “(3) a” can be used for the introduction of the epoxy group or isocyanate group, and also “(3) b” can be used for the introduction of the double bond.

In a case where a coating polymer is used, it should be understood that “substance to which a target substance can bind” or “functional group to which a target substance can bind” is immobilized on the body surfaces of the particles of the present invention and/or the surface of the coating polymer.

In a case where a coating polymer is provided on the surfaces of the bodies of the precursor particles, there is no restriction on the technique for immobilizing “functional group to which a target substance can bind”. That is to say, any suitable techniques may be used as long as “functional group to which a target substance can bind” is allowed to attach or adhere to the bodies of the precursor particles. Furthermore, “functional group to which a target substance can bind” may be immobilized prior to a provision of a coating polymer, during a provision of a coating polymer, or subsequent to a provision of a coating polymer.

In a case where a coating polymer is provided on the surfaces of the bodies of the precursor particles, an example of the technique for immobilizing “functional group to which a target substance can bind” includes a technique wherein a monomer having “functional group to which a target substance can bind” is polymerized or copolymerized during a polymerization reaction of a polymer to be provided. Examples of the monomer having “functional group to which a target substance can bind” include (meth)acrylic acid, glycidyl(meth)acrylate, hydroxyalkyl(meth)acrylate, dimethylaminoalkyl(meth)acrylate, isocyanatoalkyl (meth)acrylate, p-styrenesulfonic acid (salt), dimethylolpropanoic acid, N-alkyldiethanolamine, (aminoethylamino)ethanol and lysine.

When “functional group having stronger binding properties to a target substance” is immobilized, and also a coating polymer is provided on the surfaces of the bodies of the precursor particles, a compound may be additionally introduced into particles, the compound having two functional groups being “functional group b having reactivity with a functional group a introduced into the coating polymer by the above-described method” and “functional group c having higher binding properties to a target substance”. In this case, particles with “functional group c having higher binding properties to a target substance” immobilized thereon can be obtained by binding “functional group a” and “functional group b” to each other. When it is required to make a space between the surface of the coating polymer and “functional group to which a target substance can bind” or to make a space between the surface of the precursor particle body and “functional group to which a target substance can bind” (namely, it is required to introduce a “linker”), a compound having two functional groups being “functional group b having reactivity with the introduced functional group a” and “functional group to which a target substance can bind” may be additionally introduced into the particles with “functional group a” introduced thereto. Even in this case, “functional group to which a target substance can bind” is immobilized on particles via a bond between “functional group a” and “functional group b”. The linker may be more extended by repeating the introduction of the compound two or more times. When the space between the surface of the coating polymer and “functional group to which a target substance can bind” further increases, or the space between the surface of the precursor particle body and “functional group to which a target substance can bind” further increases, it is expected to provide an advantageous effect. For example, the degree of freedom of “functional group to which a target substance can bind” increases and thus the reactivity is improved. In addition, the degree of freedom of the target substance increases and thus the function of the target substance is not inhibited. If the number of atoms existing from a backbone of the coating polymer to the functional group is defined as the length of a linker, the above advantageous effect can be expected when the length of the linker is in the range of 5 atoms to 50 atoms. It is particularly preferred that a biogenic-related substance having a low nonspecific adsorptivity (for example, a polyethylene glycol chain) is used as a backbone of the linker.

In a case where a coating polymer is provided on the surfaces of the bodies of the precursor particles. There is no restriction on the technique for immobilizing “substance to which a target substance can bind”. That is to say, any suitable techniques may be used as long as “substance to which a target substance can bind” is allowed to attach or adhere to the precursor particle body. As with the case of “functional group to which a target substance can bind”, “substance to which a target substance can bind” may be immobilized prior to a provision of a coating polymer, during a provision of a coating polymer, or subsequent to a provision of a coating polymer.

“Substance to which a target substance can bind” can be immobilized on the precursor particles by the method similar to the above technique for introducing “functional group to which a target substance can bind”. For example, a functional group having binding properties to “substance to which a target substance can bind” is preliminarily introduced onto the surfaces of the precursor particle bodies or the surface of the coating polymer, and then “substance to which a target substance can bind” can be immobilized to the particles via the preliminarily introduced functional group. When not only the coating polymer but also “substance to which a target substance can bind” is hydrophobic, a so-called “hydrophobic interaction” can occur in water so that they are adsorbed with each other. In this way, the hydrophobic “substance to which a target substance can bind” can be immobilized on the surface of a coating polymer.

Subsequent to the step (II), it is preferable to subject the resulting particles to a washing treatment, filtering treatment or drying treatment. The washing treatment of the particles can remove impurities from the surface of each of the particles. The washing treatment of the particles is preferably performed by rising them with water. However, any suitable liquid other than the water may also be used. For example, alcoholic solvents such as ethanol, methanol and various organic solvents such as toluene and hexane can be used for the washing treatment of the particles. The filtration treatment can be performed together with the washing treatment in order to remove the washing solution from the washed particles. It is preferable to perform the drying treatment of the particles under a temperature condition of 10 to 150° C., more preferably of 40 to 90° C. The drying treatment may be performed with a dryer, but it can be nevertheless performed by a natural drying.

Hereinafter, a separation method using the particles of the present invention will be described in detail.

This separation method is intended for separating a target substance from a sample by the use of the particles of the present invention, or intended for obtaining particles with a target substance immobilized thereon. The separation method of the present invention comprises the steps of:

(i) bringing particle(s) and a sample containing a target substance(s) into contact with each other in order to bind the particle(s) and the target substance(s) to each other;

(ii) allowing the sample to stand in order to allow a spontaneous sedimentation of the particle(s) in the sample; and

(iii) recovering the particle(s) which has precipitated in the sample in order to separate the target substance(s) from the sample or obtain the particle(s) with the target substance(s) immobilized thereon.

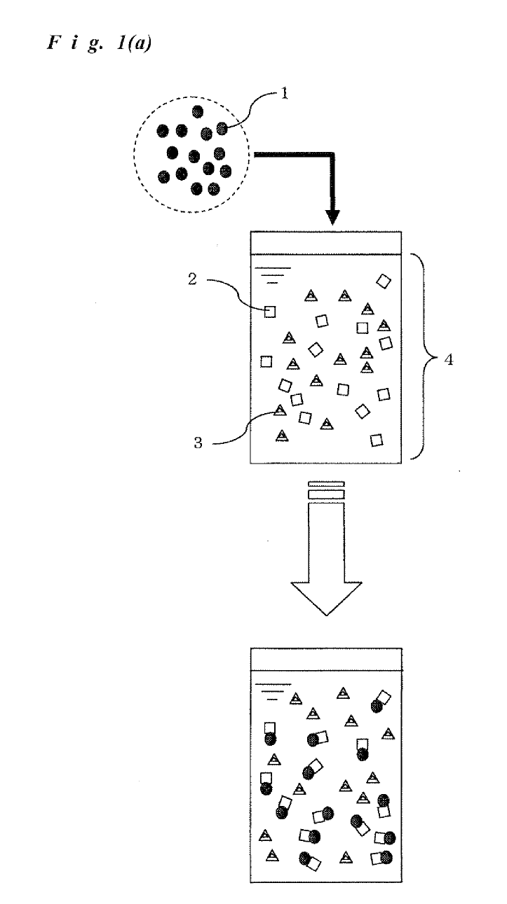

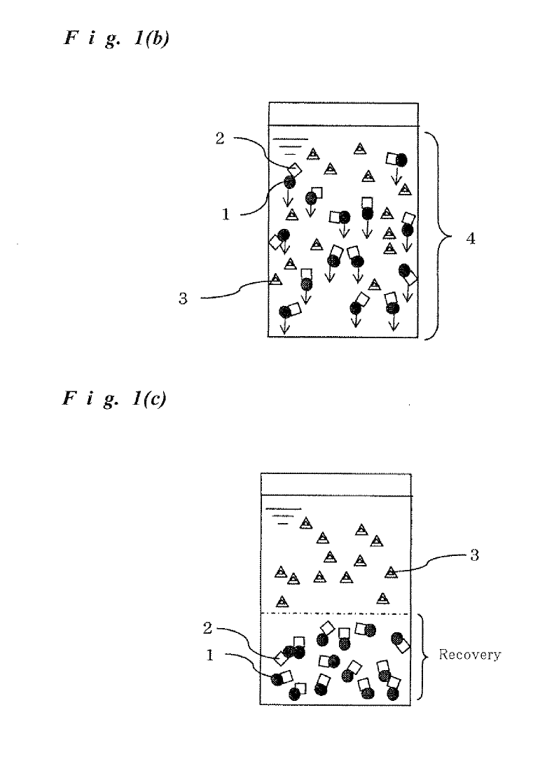

In the step (i), the particles of the present invention are brought into contact with the sample containing the target substance, and thereby the particles and the target substance are allowed to bind to each other (see FIG. 1(a)). In this regard, the sample and the particles are allowed to be in contact with each other by supplying the particles to the sample containing the target substance. If necessary, a stirring operation may be performed in order to promote the binding of the target substance to the particles. The particles to be supplied are the above-mentioned particles of the present invention (i.e. the surface-roughened particles having a form of powder preferably with an average size of 1 μm to 1 mm). The amount of the particles in powder form varies depending on the kind of samples and separation applications. For example, only one particle may be used, but the amount of particles is usually up to in gram weight (i.e. from about 10⁻² g to 10³ g) for analytical and laboratory applications, whereas the amount of particles is from in kilogram weight (i.e. about 1 to 10³ kg) to in ton weight (i.e. about 1 to 10 ton) for industrial applications.

In order to ensure the spontaneous sedimentation of the particles in the step (ii), the sample containing the target substance is preferably used in a state of being filled in a beaker, a measuring cylinder, a test tube, a microtube, a biochip, a chemical chip or a μ-TAS chip.

The binding between the target substance and the particles is brought about by an adsorptive power or affinity acting between them. More specifically, the target substance and the particles can bind to each other by the action of an adsorptive power or affinity between the target substance and “substance or functional group capable of binding to a target substance” immobilized on the particle. Depending on the amount of the particles to be supplied in powder form into the sample, there may exist particles which do not contribute to the binding of the target substance (particularly when an excessive amount of the particles are supplied). The particles to be used in the method of the present invention is characterized in that (1) the specific surface area thereof is not too large and (2) there exists a certain volume of the desirable-sized micropores, and thereby suppressing a nonspecific binding phenomenon in which “substances other than target substances” bind to the particles. Therefore, even when “substances other than target substances” are contained in the sample, the target substances can preferentially bind to the particles.

As described above, examples of the target substance include nucleic acids, proteins (e.g. avidin, biotinylated HRP and the like), sugars, lipids, peptides, cells, eumycetes(fungus), bacteria, yeasts, viruses, glycolipids, glycoproteins, complexes, inorganic substances, vectors, low molecular compounds, high molecular compounds, antibodies and antigens. As described above, examples of the sample include body fluids such as urine, blood, serum, plasma, sperm, saliva, sweat, tears, ascitic fluids and amniotic fluids from humans or animals; suspension liquids, extraction liquids, solutions and crushed solutions of organs, hair, skin, mucous membrane, nail, bone, muscle and nervous tissue from humans or animals; suspension liquids, extraction liquids, solutions and crushed solutions of stools; suspension liquids, extraction liquids, solutions and crushed solutions of cultured cells or cultured tissues; suspension liquids, extraction liquids, solutions and crushed solutions of viruses; suspension liquids, extraction liquids, solutions and crushed solutions of fungus bodies; suspension liquids, extraction liquids, solutions and crushed solutions of soil; suspension liquids, extraction liquids, solutions and crushed solutions of plants; suspension liquids, extraction liquids, solutions and crushed solutions of food and processed food; and drainage water.

In the step (ii), the sample to which the particles have been supplied is allowed to stand in order for the particles of the present invention to spontaneously settle out in the sample (see FIG. 1(b)). Due to the fact that the particles used in the method of the present invention have the above-described density, a higher spontaneous sedimentation rate can be achieved. In other words, the particles of the present invention are high density particles, so that it is possible to achieve a satisfactory separation rate only by the spontaneous sedimentation of the particles.

In the step (iii), the particles which have precipitated in the sample are collected, and thereby the target substance is separated from the sample or the particles on which the target substance has been immobilized are obtained (see FIG. 1(c)). In this regard, the precipitated particles tend to aggregate in a lower region of the sample or a bottom region of a container due to spontaneous sedimentation, whereas a supernatant is formed in an upper region of the sample. Therefore, the precipitated particles can be recovered from the sample by withdrawing the supernatant by a sucking operation using a pipette. Due to the fact that the target substance has bound to the recovered particles, the recovery of the particles can bring about a separation of the target substance from the sample.

Each of the particles to be used has the increased surface area attributable to the surface-roughening treatment, and thus has a large number of “substances or functional groups capable of binding to a target substance” immobilized thereon. Accordingly, upon the spontaneous sedimentation of the particles, the bound amount of the target substances to the particles is increased, the bound amount being per one particle. As a result, larger amount of target substances can be separated from the sample in a single treatment procedure, or the particles to which a larger amount of target substance are immobilized in a single treatment procedure. This leads to an increase of the detectable amount of the target substance as a whole, and thereby advantageous effects including an improvement of detection sensitivity, a simplified measurement and a reduction of the measurement error can be provided.

In this way, according to the method of the present invention, the target substance can be separated or the particles with the target substance immobilized thereon can be obtained. By putting this method to practice use, a system performing analysis, extraction, purification and reaction of various target substances (e.g. cells, proteins, nucleic acids and chemical substances) can be realized. More specifically, the method of the present invention makes it possible to provide a system for performing separation and immobilization of the target substances, and also to provide a system for performing analysis, extraction, purification or reaction of target substances. For example, in a system for performing analysis of the target substances, a quantitative analysis or a qualitative analysis of the target substance can be performed by using a chip wherein particles, on which an antibody combinable to the target substance is immobilized, are charged, and introducing the target substance into the chip, and thereby the target substance is immobilized to the particles within the chip, and then the amount of the target substance is detected with extinction, chemiluminescence, fluorescence, or magnetism by using the antibody to which an enzyme, a fluorochrome of a magnetic substance and the like, which is furthermore bound to the target substance, as a marker. In the case where the target substance is a nucleic acid, the quantitative analysis or the qualitative analysis of the target substance can be performed by using a chip wherein particles, on which a nucleic acid combinable to the target substance is immobilized, are charged, and introducing the target substance to which an enzyme or a fluorochrome is immobilized into the chip, and thereby the target substance is immobilized to the particles within the chip, and then the amount of the target substance is detected with extinction, chemiluminescence, fluorescence, or magnetism. In this regard, in each reaction stage, the reaction may be carried out in the same position or different positions of plural reaction vessels provided on the chip. Furthermore, for the purpose of performing a movement between plural reaction vessels provided on the chip, and also for the purpose of performing a stirring in each reaction vessel, a gravity is available. For “system for extracting a target substance” or “system for purifying a target substance”, subsequent to the separation of the step (iii), the target substance may be extracted or purified by the use of a substance capable of detaching or isolating the target substance from the particles, or by performing a required treatment such as heating or cooling. Furthermore, for “system for performing a reaction of a target substance”, a target substance is supplied to the chip wherein the particles with “substance capable of binding to a target substance” immobilized thereon are filled. As a result, the target substance is immobilized on the particles, and thereby the target substance is subjected to the reaction by performing a mixing, heating, stirring or ultraviolet irradiation in each of plural reaction vessels provided on the chip. In this case, for the purpose of performing a movement between plural reaction vessels provided on the chip, and also for the purpose of performing a stirring in each reaction vessel, the gravity is available. It is also possible that an enzyme or catalyst is immobilized on the particles and subsequently they are supplied into a reaction system by the force of gravity.

Although a few embodiments of the present invention have been hereinbefore described, the present invention is not limited to these embodiments. It will be readily appreciated by those skilled in the art that various modifications are possible without departing from the scope of the present invention.

For example, (1) in order to suppress a nonspecific binding or nonspecific adsorption to particles upon separation of a target substance; (2) in order to control affinity of the particles; or (3) in order to use as a base material for introducing a functional group, at least one kind of a substance selected from the group consisting of polyethylene glycol, polyvinyl alcohol, polyvinyl pyrrolidone, polyacrylic acid, poly(2-ethyl-2-oxazoline), polydimethylacrylamide, dextran, pullulan, agarose, sepharose, amylose, cellobiose, chitin, chitosan, polysaccharide, normal serum, bovine serum albumin, human serum albumin, casein, skimmilk powder and functional group derivatives thereof may be provided on the surface of the precursor particle body. The technique for providing the above substance is not limited, and any suitable conventional techniques for coating particles may be used. In this case, in a case where polyethylene glycol is used for example, the immobilized “substance or functional group capable of binding to a target substance” and the polyethylene glycol are present on the surface of the particle body.

It should be noted that the present invention as described above includes the following aspects:

First aspect: A particle to which a target substance can bind, characterized in that “substance or functional group capable of binding to the target substance” is immobilized on a surface of a particle body thereof; and

the surface of the particle body is a roughened surface and a specific surface area of the particle is 1.4 to 100 times a specific surface area of a true spherical particle having the same particle size and the same density as the particle of the present invention.

Second aspect: The particle according to First aspect characterized in that the surface of the particle body is the roughened surface wherein a ratio of an accumulated micropore volume [cm³] of micopores having radius of not less than 20 nm per unit surface area [cm²] is not less than 1×10⁻⁶ [cm³/cm²].

Third embodiment: The particle according to First or Second aspect characterized in that the particle has a density in the range of 3.5 g/cm³ to 9.0 g/cm³.

Fourth aspect: The particle according to any one of First to Third aspects characterized in that the particle body thereof has no through-pore.

Fifth aspect: The particle according to any one of First to Fourth aspects characterized in that the particle has the particle size in the range of 1 μm to 5 mm.

Sixth aspect: The particle according to any one of First to Fifth aspects characterized in that the particle body is made of at least one kind of a material selected from the group consisting of zirconia, yttrium-doped zirconia, iron oxide and alumina.

Seventh aspect: The particle according to any one of First to Sixth aspects characterized in that the particle exhibits magnetism.

Eighth aspect: The particle according to Seventh aspect characterized in that a saturation magnetization is in the range of 0.5 to 85 A·m²/kg.

Ninth aspect: The particle according to any one of First to Eighth aspects characterized in that a coating of polymer is provided on a part of the surface of the particle body; and

“substance or functional group capable of binding to the target substance” is immobilized on the surface of the particle body or a surface of the polymer.

Tenth aspect: The particle according to any one of First to Eighth aspects characterized in that a coating of polymer is provided over an entire surface of the particle body; and

“substance or functional group capable of binding to the target substance” is immobilized on the surface of the polymer.

Eleventh aspect: The particle according to Ninth or Tenth aspect characterized in that the polymer is at least one kind of a polymer selected from the group consisting of polystyrene, poly(meth)acrylic acid, poly(meth)acrylic acid ester, polyvinylether, polyurethane, polyamide, polyvinyl acetate, polyvinyl alcohol, polyallylamine and polyethylene imine.

Twelfth aspect: The particle according to any one of Ninth to Eleventh aspects characterized in that the polymer is a crosslinked polymer.

Thirteenth aspect: The particle according to any one of Ninth to Twelfth aspects characterized in that a silicon-containing substance and/or polyethylene glycol is present on at least a part of the surface of the particle body and/or the surface of the polymer.

Fourteenth aspect: The particle according to any one of First to Thirteenth aspects characterized in that “substance capable of binding to the target substance” is at least one kind of a substance selected from the group consisting of biotin, avidin, streptavidin and neutravidin.

Fifteenth aspect: The particle according to any one of First to Thirteenth aspects characterized in that “functional group capable of binding to the target substance” is at least one kind of a functional group selected from the group consisting of carboxyl group, hydroxyl group, epoxy group, tosyl group, succinimide group, maleimide group, thiol group, thioether group, disulfide group, aldehyde group, azido group, hydrazide group, primary amino group, secondary amino group, tertiary amino group, imide ester group, carbodiimide group, isocyanate group, iodoacetyl group, halogen-substitution of carboxyl group and double bond.

Sixteenth aspect: The particle according to any one of First to Fifteenth aspects characterized in that the target substance can bind to the particle by an adsorptivity or affinity generated between “target substance” and “substance or functional group capable of binding to the target substance”.

Seventeenth aspect: The particle according to Sixteenth aspect characterized in that the affinity generated between “target substance” and “substance or functional group capable of binding to the target substance” is due to an electrostatic interaction, π-π interaction, π-cation interaction, dipolar interaction, hydrophobic interaction, hydrogen bond, coordinate bond or biochemical interaction.

Eighteenth aspect: A method for producing a particle to which a target substance can bind, comprising the steps of:

(I) contacting precursor particle with at least one kind of acidic substance selected from the group consisting of hydrochloric acid, sulfuric acid and nitric acid (except for phosphoric acid); and

(II) immobilizing “substance or functional group capable of binding to a target substance” to the precursor particle,

wherein, in the step (I), the surface of the precursor particle is roughened so that a specific surface area of the particle is 1.4 to 100 times a specific surface area of a true spherical particle having the same particle size and the same density as those of the particle of the present invention.

Nineteenth aspect: The method according to Eighteenth aspect characterized in that, in the step (I), the precursor particle is roughened so as to have a ratio of an accumulated micropore volume [cm³] of micropores having radius of not less than 20 nm per unit surface area [cm²] is not less than 1×10⁻⁶ [cm³/cm²].

Twentieth aspect: The method according to Eighteenth or Nineteenth aspect characterized in that, in the step (I), the precursor particle is roughened so as not to produce a through-pore therein.

Twenty-first aspect: The method according to any one of Eighteenth to Twentieth aspects characterized in that, in the step (I), a mixture containing the precursor particle and the acidic substance is subjected to a hydrothermal reaction.

Twenty-second aspect: The method according to any one of Eighteenth to Twenty-first aspects characterized in that a particle with its density of 3.5 g/cm³ to 9.0 g/cm³ is used as the precursor particle.

Twenty-third aspect: A method for separating a target substance from a sample or obtaining a particle with a target substance immobilized thereon, by the use of the particle according to any one of First to Seventeenth aspects, comprising the steps of:

(i) bringing particle(s) and a sample containing a target substance(s) into contact with each other in order to bind the particle(s) and the target substance(s) to each other;

(ii) allowing the sample to stand in order to allow a spontaneous sedimentation of the particle(s) in the sample; and

(iii) recovering the particle(s) which has precipitated in the sample in order to separate the target substance(s) from the sample or obtain the particle(s) with the target substance(s) immobilized thereon.

### EXAMPLES

### Preparation of Particles

In Examples 1 to 7 and Comparative Examples 1 and 2, particles were prepared in the following manner.

### Example 1

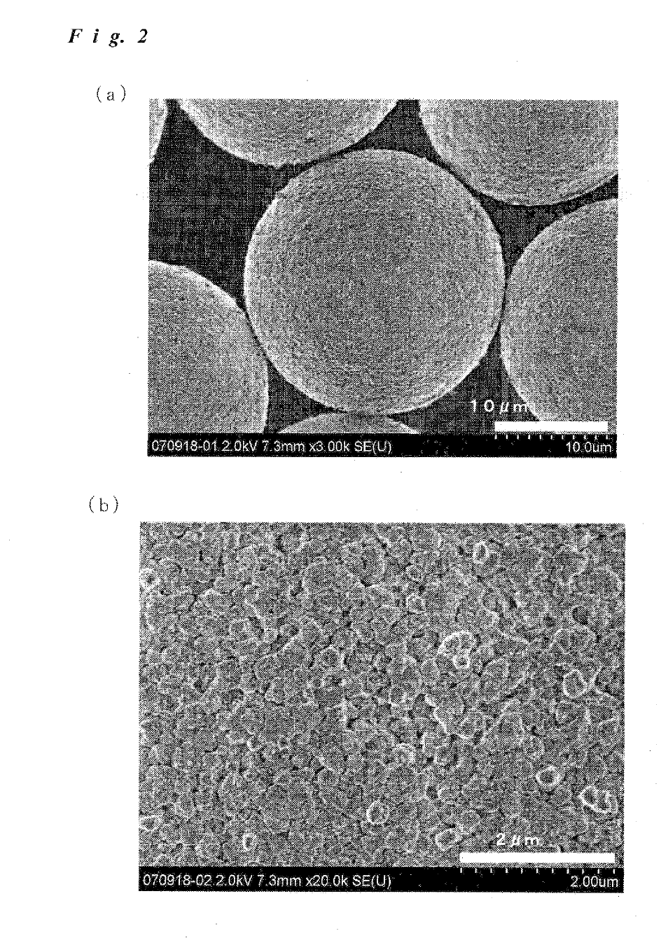

Yttrium-doped zirconia particles p1 (available from Niimi Inc.) were used. The particles p1 had a particle size of 23 μm, a specific surface area of 0.056 m²/g and a density of 6 g/cm³. The particles p1 and 25 vol % aqueous sulfuric acid solution were mixed with each other within a pressure tight vessel, and the resultant mixture was heated in a thermostatic bath at a temperature of 200° C. for 6 hours. Thereafter, the mixture was washed and dried. After the above procedures, it was confirmed that the specific surface area of the resultant particles was 0.40 m²/g. An electron micrograph of such particles is shown in FIG. 2 wherein FIG. 2(a) is an overall view of the particle, and FIG. 2(b) is an enlarged view of the surface of the particle. Subsequently, 10 g of the particles were dispersed into 25 g of pure water and then 3 g of 3-glycidoxypropyltrimethoxysilane was added into the resultant dispersion while stirring, followed by further stirring for 4 hours. After washing particles with acetone, the particles were subjected to a vacuum drying treatment, and thereby the yttrium-doped zirconia particles with an epoxy group thereon were obtained. Subsequently, an aqueous solution prepared by dissolving 5 mg of avidin in 1 ml of 10 mM PBS solution (pH 7.2) was added to 100 mg of the resultant particles, followed by stirring overnight. After washing the particles with a 10 mM PBS solution (pH 7.2) and water, the particles were subjected to a vacuum drying treatment, and thereby the yttrium-doped zirconia particles P1 with avidin immobilized thereon were obtained. The particles P1 had a particle size of 23 μm, a specific surface area of 0.40 m²/g and a density of 6 g/cm³. The specific surface area 0.40 m²/g of the particles P1 was 9.2 times larger than the specific surface area of the true spherical particle having a smooth surface and having the particle size 23 μm (i.e. the specific surface area 0.043 m²/g obtained from the particle size 23 μm and the density 6 g/m³). The accumulated micropore volume of the particles regarding micropores having radius of not less than 20 nm was 3.3×10⁻³ cm³/g. Thus, the ratio of the accumulated micropore volume regarding micropores having radius of not less than 20 nm per unit surface area [cm²] of the true spherical particle having the same particle size and the same density as the obtained particles P1 was 7.6×10⁻⁶ [cm³/cm²].

It should be noted that, although the above Example 1 used a silane coupling agent having an epoxy group, other suitable silane coupling agents having other functional groups such as amino group, isocyanate group, mercapto group and double bond may also be used instead.

### Example 2

The same procedure as that of Example 1 was performed except for the conditions of the sulfuric acid treatment in Example 2 being a temperature of 200° C. and treatment time of 8 hours. The obtained particles P2 in Example 2 had a particle size of 23 μm, a specific surface area of 1.6 m²/g and a density of 6 g/cm³. The specific surface area 1.6 m²/g of the particles P2 was 37 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles P2 regarding micropores having radius of not less than 20 nm was 8.3×10⁻³ cm³/g. Thus, the ratio of the accumulated micropore volume regarding micropores having radius of not less than 20 nm per unit surface area [cm²] of the true spherical particle having the same particle size and the same density as the obtained particles P2 was 1.9×10⁻⁵ [cm³/cm²].

### Example 3

The same procedure as that of Example 1 was performed except for the conditions of the sulfuric acid treatment in Example 3 being a temperature of 200° C. and treatment time of 12 hours. The obtained particles P3 in Example 3 had a particle size of 23 μm, a specific surface area of 2.7 m²/g and a density of 6 g/cm³. The specific surface area 2.7 m²/g of the particles P3 was 62 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles P3 regarding micropores having radius of not less than 20 nm was 2.6×10⁻² cm³/g. Thus, the ratio of the accumulated micropore volume regarding micropores having radius of not less than 20 nm per unit surface area [cm²] of the true spherical particle having the same particle size and the same density as the obtained particles P3 was 6.0×10⁻⁵ [cm³/cm²].

### Example 4

The same procedure as that of Example 1 was performed except for the conditions of the sulfuric acid treatment in Example 4 being a temperature of 200° C. and treatment time of 16 hours. The obtained particles P4 in Example 4 had a particle size of 23 μm, a specific surface area of 3.9 m²/g and a density of 6 g/cm³. The specific surface area 3.9 m²/g of the particles P4 was 90 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles P4 regarding micropores having radius of not less than 20 nm was 6.3×10⁻² cm³/g. Thus, the ratio of the accumulated micropore volume regarding micropores having radius of not less than 20 nm per unit surface area [cm²] of the true spherical particle having the same particle size and the same density as the obtained particles P4 was 1.4×10⁻⁴ [cm³/cm²].

### Example 5

The same procedure as that of Example 1 was performed except for the conditions of the sulfuric acid treatment in Example 5 being a temperature of 160° C. and treatment time of 6 hours. The obtained particles P5 in Example 5 had a particle size of 23 μm, a specific surface area of 0.12 m²/g and a density of 6 g/cm³. The specific surface area 0.12 m²/g of the particles P5 was 2.8 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles P5 regarding micropores having radius of not less than 20 nm was 7.2×10⁻⁴ cm³/g. Thus, the ratio of the accumulated micropore volume regarding micropores having radius of not less than 20 nm per unit surface area [cm²] of the true spherical particle having the same particle size and the same density as the obtained particles P5 was 1.7×10⁻⁶ [cm³/cm²].

### Example 6

The same procedure as that of Example 1 was performed except for the following matters: In Example 6, the particles were mixed with 25 vol % nitric acid solution instead of the sulfuric acid treatment, and the condition of the heating treatment by the thermostatic bath was a temperature of 200° C. and treatment time of 4 hours. The obtained particles P6 in Example 6 had a particle size of 23 μm, a specific surface area of 0.50 m²/g and a density of 6 g/cm³. The specific surface area 0.50 m²/g of the particles P6 was 12 times larger than the specific surface area of the true spherical particle having a smooth surface and having the particle size 23 m (i.e. the specific surface area 0.043 m²/g obtained from the particle size 23 μm and the density 6 g/m³).

The accumulated micropore volume of the particles P6 regarding micropores having radius of not less than 20 nm was 4.1×10⁻³ cm³/g. Thus, the ratio of the accumulated micropore volume regarding micropores having radius of not less than 20 nm per unit surface area [cm²] of the true spherical particle having the same particle size and the same density as the obtained particles P6 was 9.4×10⁻⁶ [cm³/cm²].

### Example 7

The same procedure as that of Example 1 was performed except for the following matters: In Example 7, the heating treatment was performed at a temperature of 200° C. for 2 hours by means of a microwave instead of the sulfuric acid treatment. The obtained particles P7 in Example 7 had a particle size of 23 μm, a specific surface area of 0.45 m²/g and a density of 6 g/cm³. The specific surface area 0.45 m²/g of the particles P7 was 10 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles P7 regarding micropores having radius of not less than 20 nm was 3.4×10⁻³ cm³/g. Thus, the ratio of the accumulated micropore volume regarding micropores having radius of not less than 20 nm per unit surface area [cm²] of the true spherical particle having the same particle size and the same density as the obtained particles P7 was 7.8×10⁻⁶ [cm³/cm²].

### Comparative Example 1

The same procedure as that of Example 1 was performed except for no sulfuric acid treatment being performed in Comparative Example 1. The obtained particles R1 in Comparative Example 1 had a particle size of 23 μm, a specific surface area of 0.056 m²/g and a density of 6 g/cm³. The specific surface area 0.056 m²/g of the particles R1 was 1.3 times larger than the specific surface area of the true spherical particle having a smooth surface and having the particle size 23 μm (i.e. the specific surface area 0.043 m²/g obtained from the particle size 23 μm and the density 6 g/m³).

The accumulated micropore volume of the particles R1 regarding micropores having radius of not less than 20 nm was 7.5×10⁻⁵ cm³/g. Thus, the ratio of the accumulated micropore volume regarding micropores having radius of not less than 20 nm per unit surface area [cm²] of the true spherical particle having the same particle size and the same density as the obtained particles R1 was 1.7×10⁻⁷ [cm³/cm²]. An electron micrograph of the particle of Comparative Example 1 is shown in FIG. 3 wherein FIG. 3(a) shows a whole particle and FIG. 3(b) shows an enlarged view of the surface of the particle.

### Comparative Example 2

The same procedure as that of Example 1 was performed except for the following matters: In Comparative Example 2, the porous zirconia particles with through-pores therein were used as the precursor particles, and no sulfuric acid treatment was performed. The obtained particles R2 in Comparative Example 2 had a particle size of 25 μm, a specific surface area of 17.0 m²/g and a density of 6 g/cm³. The specific surface area 17.0 m²/g of the particles R2 was 425 times larger than the specific surface area of the true spherical particle having a smooth surface and having the particle size 25 μm (i.e. the specific surface area 0.040 m²/g obtained from the particle size 25 μm and the density 6 g/m³).

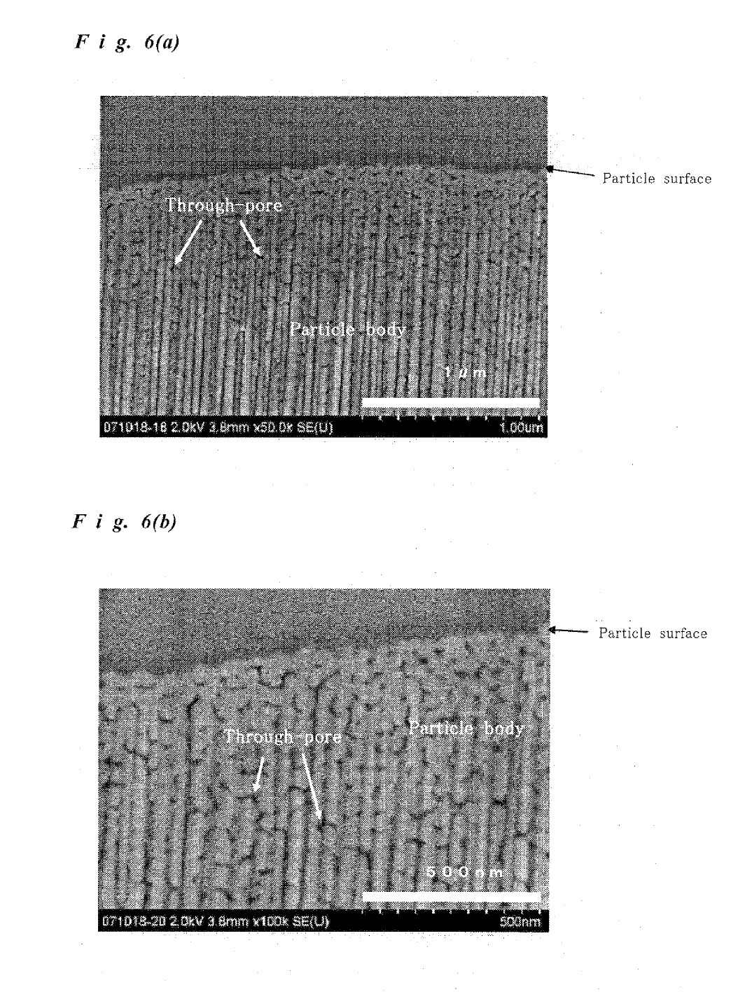

The accumulated micropore volume of the particles R2 regarding micropores having radius of not less than 20 nm was 4.0×10⁻⁴ cm³/g. Thus, the ratio of the accumulated micropore volume regarding micropores having radius of not less than 20 nm per unit surface area [cm²] of the true spherical particle having the same particle size and the same density as the obtained particles R2 was 1.0×10⁻⁶ [cm³/cm²]. An electron micrograph of the particles of Comparative Example 2 is shown in FIG. 3 wherein FIG. 6(a) shows a whole particle and FIG. 6(b) shows an enlarged view of the surface of the particle.

The conditions of the treatment and the result in Examples 1-7 and Comparative Examples 1-2 are summarized in Table 1.

TABLE 1

| | | | | | | | | | | Accumulated Volume of | |
|---|---|---|---|---|---|---|---|---|---|---|---|
| | | | | | | | | | | micropores having radius | |
| | | | | | | | | BET | | of not less than 20 nm | |
| | | Particle | | Acid Treatment | | | | Measured | | Measured | |
| | | Size | Density | | | Temperature | Time | value | Factor against | Value | per Unit Area |
| Example | Particle | [μm] | [g/cm³] | Heating | Acid | [° C.] | [h] | [m²/g] | Theoretical Value* | [cm³/g] | [cm³/cm²] |
| Example 1 | P1 | 23 | 6 | Thermostatic | Sulfuric | 200 | 6 | 0.40 | 9.2 | 3.3 × 10⁻³ | 7.6 × 10⁻⁶ |
| | | | | Bath | Acid | | | | | | |
| Example 2 | P2 | 23 | 6 | Thermostatic | Sulfuric | 200 | 8 | 1.6 | 37 | 8.3 × 10⁻³ | 1.9 × 10⁻⁵ |
| | | | | Bath | Acid | | | | | | |
| Example 3 | P3 | 23 | 6 | Thermostatic | Sulfuric | 200 | 12 | 2.7 | 62 | 2.6 × 10⁻² | 6.0 × 10⁻⁵ |
| | | | | Bath | Acid | | | | | | |
| Example 4 | P4 | 23 | 6 | Thermostatic | Sulfuric | 200 | 16 | 3.9 | 90 | 6.3 × 10⁻² | 1.4 × 10⁻⁴ |
| | | | | Bath | Acid | | | | | | |
| Example 5 | P5 | 23 | 6 | Thermostatic | Sulfuric | 160 | 6 | 0.12 | 2.8 | 7.2 × 10⁻⁴ | 1.7 × 10⁻⁶ |
| | | | | Bath | Acid | | | | | | |
| Example 6 | P6 | 23 | 6 | Thermostatic | Nitric | 200 | 4 | 0.50 | 12 | 4.1 × 10⁻³ | 9.4 × 10⁻⁶ |
| | | | | Bath | Acid | | | | | | |
| Example 7 | P7 | 23 | 6 | Microwave | Sulfuric | 200 | 2 | 0.45 | 10 | 3.4 × 10⁻³ | 7.8 × 10⁻⁶ |
| | | | | | Acid | | | | | | |

| | | | | | | | | |
|---|---|---|---|---|---|---|---|---|
| Comparative | R1 | 23 | 6 | [symbol] | 0.056 | 1.3 | 7.5 × 10⁻⁵ | 1.7 × 10⁻⁷ |
| Example 1 | | | | | | | | |
| Comparative | R2 | 25 | 6 | [symbol] | 17 | 425 | 4.0 × 10⁻⁴ | 1.0 × 10⁻⁶ |
| Example 2 | | | | | | | | |

(*Ratio of the specific surface area of the particle to the specific surface area of the true spherical particle)

### <<Confirmation of Roughness and Confirmation of Structure of Particle Body>>

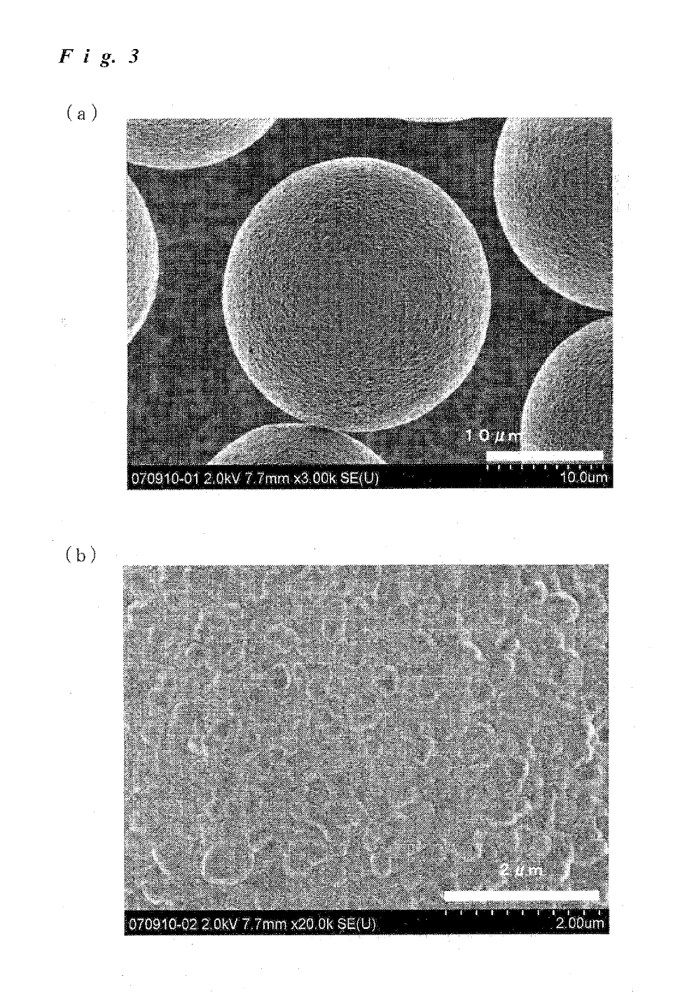

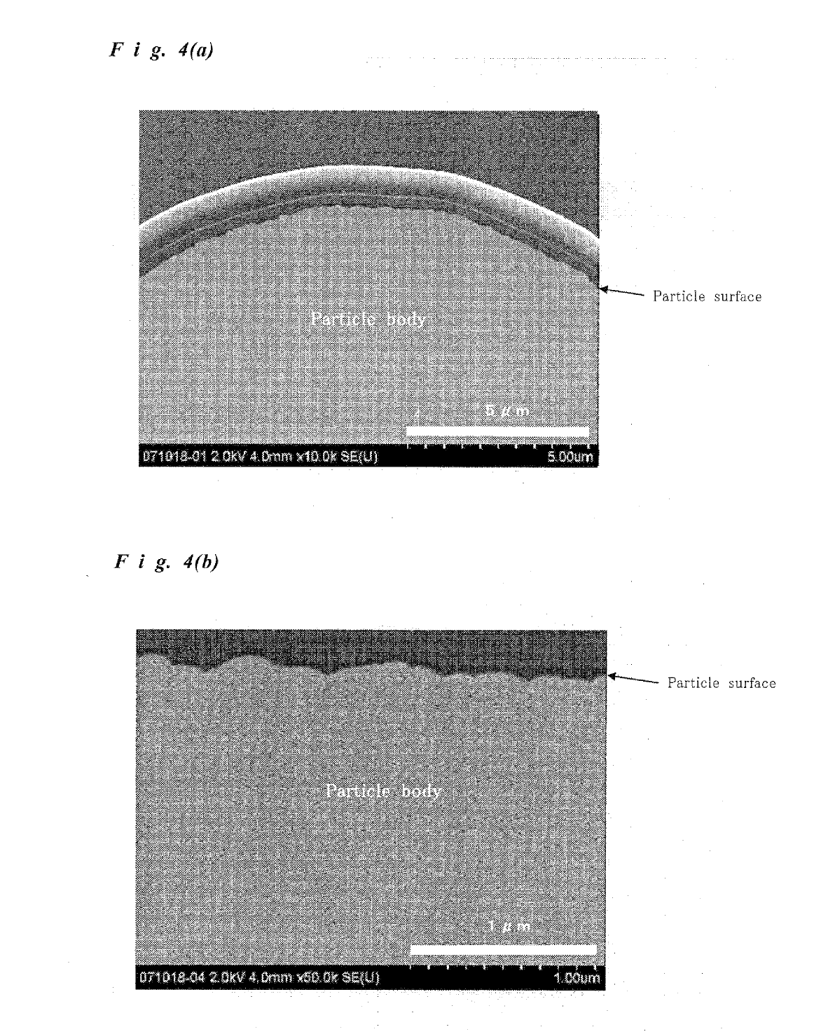

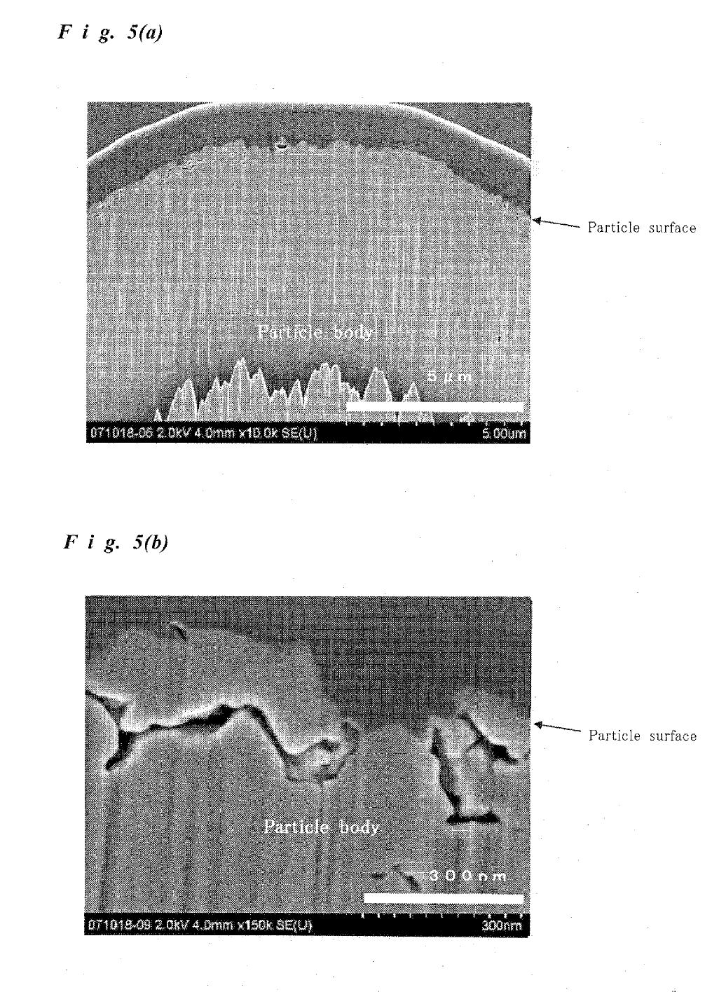

The surface change of the particle after the above sulfuric acid treatment was confirmed by the images. Each of FIGS. 4(a) and 4(b) shows cross-section of the precursor particle p1 at the vicinity of the surface thereof in Example 1 (i.e. the particle before being subjected to the sulfuric acid treatment). On the other hand, each of FIGS. 5(a) and 5(b) shows cross-section of the precursor particle p1 at the vicinity of the surface thereof in Example 1 after being subjected to the sulfuric acid treatment. Comparing the image of FIG. 4(b) to that of FIG. 5(b), it can be understood that the particle has become to have an indented surface by the above sulfuric acid treatment, and thus the surface of the particle has been roughened.

Each of FIGS. 6(a) and (b) shows a cross-section of the porous zirconia particle at the vicinity of the surface thereof. In FIGS. 6(a) and (b), “black portions having an undulating form” show the through-pores of the particle, and thus it was confirmed that the particle was porous particle. In contrast, such “black portions having an undulating form” do not exist in the image of the precursor particle of Example 1 as shown in FIGS. 4(a) and (b), and thus indicating that the body of the precursor particle in Example 1 had no through-pore.

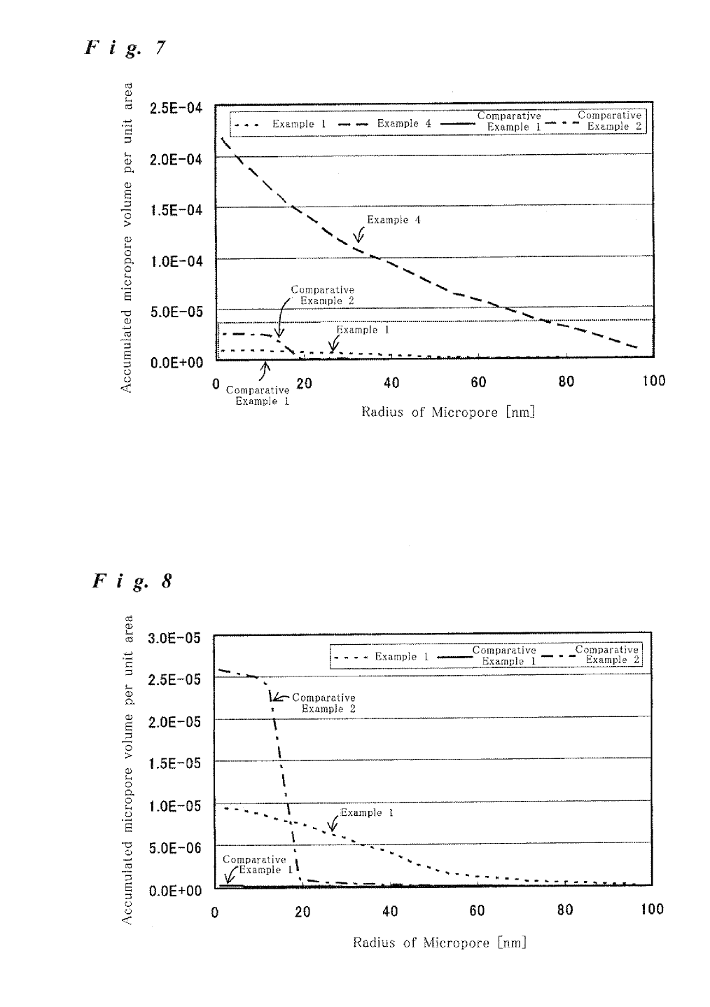

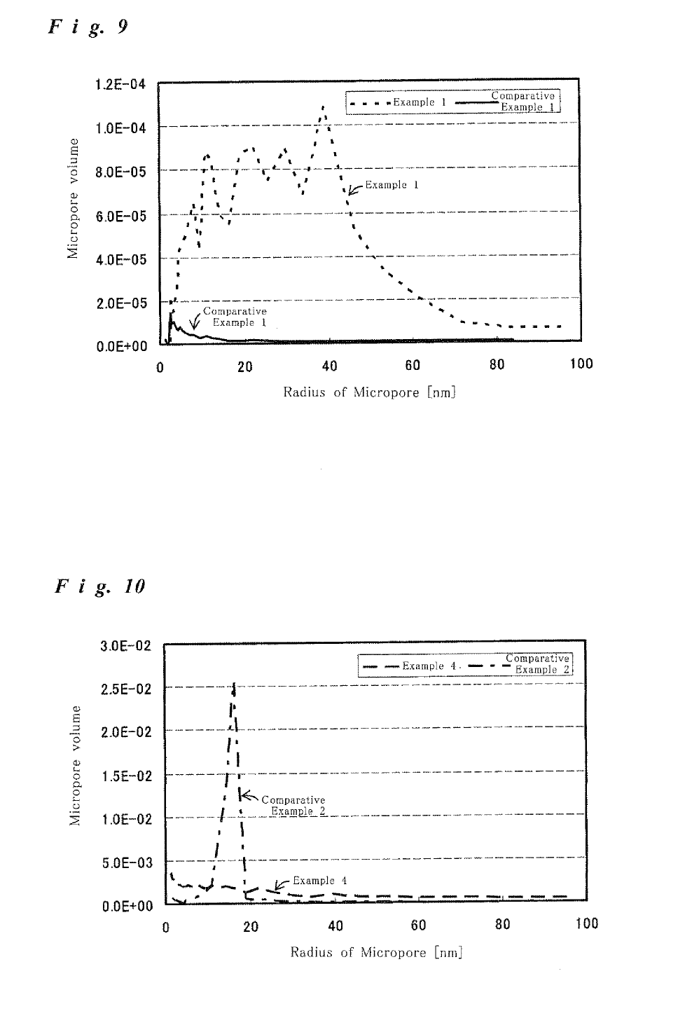

FIGS. 7-10 show graphs showing a relationship between the micropore radius and the accumulated micropore volume. Each of FIGS. 7 and 8 shows the relationship between “micropore radii” and “accumulated micropore volume obtained by integrating the volumes of the micropores having radius of no more than 100 nm from 100 nm side”. Each of FIGS. 9 and 10 shows the relationship between “micropore radius of the particle” and “micropore volume at each micropore radius”. These two kinds of graphs show substantially the same matter. It has been found that the particle of each of Examples 1 and 4 has a larger accumulated micropore volume than that of Comparative Example 1. It has been also found that the particle of Comparative Example 2 has the extremely large value of the accumulated micropore volume regarding micropores having radius of no more than 20 nm as shown in FIG. 8 (namely, the particle has a lot of micropores with radius of no more than 20 nm), and also has a narrow size distribution of the micropore as shown in FIG. 10.

### <<Confirmation of Binding Property to Target Substance>>

The binding property of the particles with respect to the target substance was confirmed using particle P1 obtained from Example 1, particle R1 obtained from Comparative Example 1 and particle R2 obtained from Comparative Example 2. Biotinylated HRP was used as the target substance. In general, avidin immobilized on the particle specifically binds to the biotinylated HRP.

The same procedure was applied to the particles P1, R1 and R2 of the Example 1, Comparative Examples 1 and 2 to study the binding ability between biotinylated HRP and the particle among the particles P1, R1 and R2. First, three 1.5 ml-tubes were prepared and an appropriate amount (which enables a chromogenic amount of 0.01 to 1.5) of particles were respectively charged thereinto. After adding 100 μl of 20 ng/ml biotinylated HRP to the tubes respectively, the contents of the tubes were respectively stirred with a Voltex mixer for 30 minutes. Thereafter, the particles charged in each tube was washed with 500 μl of a 10 mM PBS buffer solution (pH 7.2) four times. After the PBS buffer solution (pH 7.2) was removed, 200 μl of TMB (tetramethylbenzidine) was added to each tube containing the particles, followed by standing for 30 minutes, and thereby causing color development of the particles. The reaction was terminated by adding 200 μl of 1N sulfuric acid. Each absorbance of the particles in each tube was obtained by measuring an absorbance (450 nm) of the supernatant fluid by means of Microplate Reader Infinite 200 manufactured by TECAN. The results are shown in Table 2. The measured values were 0.24 for the particle P1, 0.03 for the particle R1 and 0.06 for the particle R2, respectively. These results indicate that the particle P1 has a 8-fold increased binding ability than that of the particle R1 due to the increased surface area of the particle. On the other hand, the absorbance of the particle R2 has is less than half of that of P1 although the specific surface area of the particle R2 was much larger than that of particle P1. In this regard, it is contemplated that the particle R2 had the accumulated micropore volume of the micropores having radius of not less than 20 nm per unit surface area being about one-eighth of that of the particle P1, so that the micropores of the particle R2 were not effectively utilized for “functional group capable of binding to a target substance”.

TABLE 2

| | | Ratio [cm³/cm²] of accumulated micropore | |
|---|---|---|---|
| | | volume [cm³] of micropores having radius of 20 | |
| Example | Particle | nm or more per unit surface area [cm²] of particle | Absorbance |
| Example 1 | P1 | 7.6 × 10⁻⁶ | 0.24 |
| Comparative | R1 | 1.7 × 10⁻⁷ | 0.03 |
| Example 1 | | | |
| Comparative | R2 | 1.0 × 10⁻⁶ | 0.06 |
| Example 2 | | | |

### <<Confirmation of Nonspecific Binding-Suppressing Effect of Surface-Roughened Particles>>

Confirmatory test on the effect of suppressing the nonspecific binding phenomenon was carried out with respect to the particles of the present invention. The object of this confirmatory test is to confirm that the surface-roughened particles of the present invention have more suppressing effect for the nonspecific binding than that of the porous particles having through-pores. Specifically, “Surface-roughened particles (particles obtained after the surface-roughening treatment in Example 1)” and “Porous particles with through-pores therein in Comparative Example 2” were respectively used to evaluate “specific binding ability” and “nonspecific binding ability”. The term “specific binding ability” means a binding ability for a target substance to bind to the particles. On the other hand, the term “nonspecific binding ability” means a binding ability for the substance other than the target substance to bind to the bodies of the particles.

### (Preparation of Particles)

The immobilization of “3-glycidoxypropyl-trimethoxysilane having a terminal epoxy group” to each of the “Surface-roughened particles P1 (Example 1)” and the “Porous particles R2 with through-pores therein (Comparative Example 2)” was performed. As a result, epoxy particles with epoxy group immobilized on the surface thereof were obtained without binding the avidin to the particle. The epoxy particles derived from the Surface-roughened particles P1 (Example 1) are referred to as “S1”, whereas the epoxy particles derived from the Porous particles R2 with through-pores therein (Comparative Example 2) are referred to as “T2”.

As the specific binding ability, a binding ability of the epoxy particles (S1 and T2) with respect to Texas Red (Trade Name: Sulforhodamine101 cadaverine available from Biotium) which has an amino group was evaluated. On the other hand, as the nonspecific binding ability, a binding ability of the epoxy particles (S1 and T2) with respect to another Texas Red (Trade Name: Sulforhodamine101*Fluorescence Reference Standard* available from ABD Bioquest, Inc.) which has no amino group was evaluated. In the evaluations of such binding abilities, the fluorescence intensities of Texas Reds of the particles were respectively measured by means of a fluorescence microscope, and then the bound amounts of Texas Reds, which had bound to the surface of the particles, were also respectively determined from the fluorescence intensities. Specifically, the procedures were as follows:

For the evaluation of the specific binding abilities, each of epoxy particles (S1 and T2) metered in an amount of 0.5 mg was introduced into an Eppendorf tube, followed by adding 50 μl of a 0.5 mg/ml aqueous solution of Sulforhodamine101 cadaverine. The resultant dispersion liquid was stirred at 1500 rpm for two hours, and thereafter the particles in the dispersion liquid were subjected to a washing treatment with 100 μl of 10 mM phosphate buffer liquid (pH 7.2) three times.

Similarly, for the evaluation of the nonspecific binding abilities, each of epoxy particles (S1 and T2) metered in an amount of 0.5 mg was introduced into an Eppendorf tube, followed by adding 50 μl of a 0.5 mg/ml aqueous solution of Sulforhodamine101*Fluorescence Reference Standard*. The resultant dispersion liquid was stirred at 1500 rpm for two hours, and thereafter the particles in the dispersion liquid were subjected to a washing treatment with 100 μl of 10 mM phosphate buffer liquid (pH 7.2) three times.

### (Evaluation of Binding Abilities)

The obtained particles (S1 and T2) had Texas Reds which had bound thereto, and thus the intensity of fluorescence emitted therefrom was measured. Specifically, image on the fluorescence emitted from Texas Reds which had been bound to the particles was taken with a CCD camera, together with a fluorescence microscope in which a filter set for Texas Red (manufactured by OPTO-LINE Inc.) was installed. Then, the intensity of fluorescence was measured through an image analysis by means of an image analysis software “Image-Pro Plus (available from Media Cybernetics, Inc.)”. As a result, the bound amount of Texas Red (Sulforhodamine101 cadaverine) having an amino group was obtained (namely, the bound amount regarding the specific binding was obtained) with respect to each of the particles S1 and T2. And also, the bound amount of Texas Red (Sulforhodamine101*Fluorescence ReferenceStandard*) having no amino group was obtained (namely, the bound amount regarding the nonspecific binding was obtained) with respect to each of the particles S1 and T2.

Upon obtaining the above bound amounts, a so-called calibration curve method was applied. Specifically, aqueous solutions wherein the concentrations of Sulforhodamine101 cadaverine were varied were prepared for “specific binding” as calibration standard solutions, whereas aqueous solutions wherein the concentrations of Sulforhodamine101*Fluorescence Reference Standard* were varied were also prepared for “nonspecific binding” as calibration standard solutions. Then, by use of the calibration curves obtained therefrom, the bound amounts were calculated from the intensities of the fluorescence.

### (Results)

The results are shown in Table 3 below. Table 3 shows the ratio of the amount of the “nonspecific binding” to the amount of the “specific binding”. That is, Table 3 shows the values of “nonspecific binding/specific binding” with respect to the particles S1 and T2. In general, when such value of “nonspecific binding/specific binding” is smaller, the amount of the nonspecific binding is smaller than that of the specific binding, and thereby indicating that the effect of the nonspecific binding is small. On the other hand, when the value of “nonspecific binding/specific binding” is larger, the amount of the nonspecific binding is larger than that of the specific binding, and thereby indicating that the effect of the nonspecific binding is large.

In these regards, the value of “nonspecific binding/specific binding” was 0.10 for the Surface-roughened particles S1, while on the other hand the value of “nonspecific binding/specific binding” was 0.32 for the porous particles T2 with through-pores therein. Accordingly, it was confirmed that the Surface-roughened particles S1 had more effect of suppressing the nonspecific binding phenomenon than that of the porous particles T2.

TABLE 3

| | Surface-roughened | Porous Particles with |
|---|---|---|
| | Particles S1 | through-pores therein T2 |
| Nonspecific Binding/ | 0.10 | 0.32 |
| Specific Binding | | |

### INDUSTRIAL APPLICABILITY

The particles of the present invention can be used for a quantitative determination, separation, purification, analysis and the like of target substances such as cells, proteins, nucleic acids and chemical substances. For example, the particles of the present invention capable of binding to nucleic acids such as DNA can be used for analysis of DNA, and thus they contribute to tailor-made medical technologies.

### CROSS REFERENCE TO RELATED PATENT APPLICATION

The present application claims a priority under the Paris Convention based on Japanese Patent Application No. 2008-158949 (filed Jun. 18, 2008, the title of the invention: “SURFACE-ROUGHENED HIGH-DENSITY FUNCTIONAL PARTICLE, METHOD FOR PRODUCING THE SAME AND METHOD FOR TREATING TARGET SUBSTANCE WITH THE SAME”), and the contents of which are incorporated herein by reference in their entirety.

1-17. (canceled)

18. A particle to which a target substance can bind, characterized in that a substance or functional group capable of binding to the target substance is immobilized on a surface of a particle body thereof; and
the surface of the particle body is a roughened surface and a specific surface area of the particle is 1.4 to 100 times a specific surface area of a true spherical particle having the same particle size and the same density as the particle, wherein a ratio of an accumulated micropore volume [cm³] of micopores having radius of not less than 20 nm per unit surface area [cm²] is not less than 1×10⁻⁶ [cm³/cm²].

19. The particle according to claim 18, characterized in that a density of the particle is in the range of 3.5 g/cm³ to 9.0 g/cm³.

20. The particle according to claim 18, characterized in that the particle body has no through-pore.

21. The particle according to claim 18, characterized in that the particle size of the particle is in the range of 1 μm to 5 mm.

22. The particle according to claim 18, characterized in that the particle body is made of at least one kind of a material selected from the group consisting of zirconia, yttrium-doped zirconia, iron oxide and alumina.

23. The particle according to claim 18, characterized in that the substance capable of binding to the target substance is at least one kind of a substance selected from the group consisting of biotin, avidin, streptavidin and neutravidin.

24. The particle according to claim 18, characterized in that the functional group capable of binding to the target substance is at least one kind of a functional group selected from the group consisting of carboxyl group, hydroxyl group, epoxy group, tosyl group, succinimide group, maleimide group, thiol group, thioether group, disulfide group, aldehyde group, azido group, hydrazide group, primary amino group, secondary amino group, tertiary amino group, imide ester group, carbodiimide group, isocyanate group, iodoacetyl group, halogen-substitution of carboxyl group and double bond.

25. The particle according to claim 18, characterized in that:
a coating of polymer is provided on a part of the surface of the particle body; and
the substance or functional group capable of binding to the target substance is immobilized on the surface of the particle body or a surface of the polymer.

26. The particle according to claim 18, characterized in that:
a coating of polymer is provided on the whole surface of the particle body; and
the substance or functional group capable of binding to the target substance is immobilized on a surface of the polymer.

27. The particle according to claim 18, characterized in that the particle is a magnetic particle.

28. The particle according to claim 18, characterized in that the target substance can bind to the particle by an adsorptivity or affinity generated between the target substance and the substance or functional group capable of binding to the target substance.

29. A method for producing a particle to which a target substance can bind, comprising the steps of:
(I) contacting a precursor particle with at least one kind of acidic substance selected from the group consisting of hydrochloric acid, sulfuric acid and nitric acid; and
(II) immobilizing a substance or functional group capable of binding to the target substance onto the precursor particle
wherein, in the step (I), the surface of the precursor particle is roughened so that a specific surface area of the particle is 1.4 to 100 times a specific surface area of a true spherical particle having the same particle size and the same density as the particle.

30. The method according to claim 29, characterized in that, in the step (I), the precursor particle is roughened so as to have a ratio of an accumulated micropore volume [cm³] of micropores having radius of not less than 20 nm per unit surface area [cm²] is not less than 1×10⁻⁶ [cm³/cm²].

31. The method according to claim 29, characterized in that, in the step (I), a mixture containing the precursor particle and the acidic substance is subjected to a hydrothermal reaction.

32. The method according to claim 29, characterized in that a particle with a density of 3.5 g/cm³ to 9.0 g/cm³ is used as the precursor particle.

33. A method for separating a target substance from a sample or obtaining a particle with a target substance immobilized thereon, by the use of the particle according to claim 1, comprising the steps of:
(i) bringing the particle and a sample containing a target substance into contact with each other, and thereby binding the particle and the target substance to each other;
(ii) allowing the sample to stand, and thereby allowing a spontaneous sedimentation of the particle in the sample; and
(iii) recovering the particle which has precipitated in the sample, and thereby separating the target substance from the sample or obtaining the particle with the target substance immobilized thereon.
